(19) United States
(12) Patent Application Publication
(10) Pub. No.: US 2025/0283822 A1
(43) Pub. Date: Sep. 11, 2025

(54) COMPOSITIONS AND METHODS FOR IMPROVED MULTIPLEXED ERROR ROBUST FLUORESCENCE IN SITU HYBRIDIZATION

(71) Applicant: Vizgen, Inc., Cambridge, MA (US)

(72) Inventors: George Emanuel, Cambridge, MA (US); Jiang He, Cambridge, MA (US); Li Zhi He, Cambridge, MA (US)

(21) Appl. No.: 19/076,666

(22) Filed: Mar. 11, 2025

Related U.S. Application Data
(60) Provisional application No. 63/563,863, filed on Mar. 11, 2024.

(51) Int. Cl. G01N 21/64; C12Q 1/6841

(52) U.S. Cl. CPC G01N 21/6456; C12Q 1/6841; G01N 21/6428; G01N 2021/6441

(57) ABSTRACT

The present disclosure is generally directed to compositions and methods for determining nucleic acid targets in biological samples via in situ imaging and improved multiplexed error robust fluorescence in situ hybridization. The disclosure provides compositions and methods generally directed to nucleic acid probes configured to hybridize to cellular nucleic acids and improve in situ imaging signals.

### RELATED APPLICATIONS

This application claims priority to Ser. No. 63/563,863 filed on 11 Mar. 2024, which is incorporated by reference into this application in its entirely.

### FIELD OF THE DISCLOSURE

This application relates generally to the field of in situ imaging, and in particular, relates to methods of determining nucleic acid targets within tissue samples.

### BACKGROUND OF THE DISCLOSURE

To accurately profile in situ gene expression in a biological sample, a spatial transcriptomics technique with high detection efficiency and spatial resolution is required. In situ single cell transcriptomic imaging technology, such as Multiplexed Error-Robust Fluorescence In Situ Hybridization (MERFISH), enables the direct profiling of the spatial organization of intact tissue with subcellular resolution. Through combinatorial labeling and sequential imaging, different RNA species are imaged by fluorescence microscopy to generate a specific pattern of fluorescent ON and OFF signal, an optical barcode that can be used to resolve the location and quantity of different RNA species. This spatially resolved RNA-profiling data gives a physical picture for the cell or tissue of interest, which can help elucidate the intricate interplay between different cell types in complex biological systems.

However, current MERFISH methods rely upon fiducial markers coating the surface of a sample slide in order to align MERFISH sequential imaging data. These fiducial markers can create bright clusters that interfere with fluorescent imaging, can be too sparse for accurate sequential image alignment, are difficult to manufacture, and can be destroyed during MERFISH sample preparation. It is thus the object of the present disclosure to provide novel compositions and methods that facilitate improved MERFISH workflows, improved fiducial signals without the need for fiducial marker-coated sample slides, improved MERFISH signal uniformity and brightness, and reduced MERFISH background signal.

### SUMMARY OF THE DISCLOSURE

Described herein are methods and reagents thereof for in situ single-cell transcriptomic analysis from biological samples.

In certain embodiments provided herein are compositions comprising a plurality of primary nucleic acid probes, each nucleic acid probe comprising a target binding sequence, one or more converter sequences complementary to a sandwich probe, wherein each unique converter sequence is assigned to two or more positions in a valid codeword assigned to a distinct nucleic acid target; wherein subpopulations of the primary nucleic acid probes hybridize to the distinct nucleic acid target and comprise two or more unique converter sequences that translate to the valid codeword for the distinct nucleic acid target.

In some embodiments, provided are compositions comprising a sandwich probe further comprises an in situ fiducial binding site. In embodiments, the sandwich probe further comprises one of more read sequences. In certain embodiments, the sandwich probe comprises a poly T sequence between the read sequence and the in situ fiducial binding site.

Provided herein in certain embodiments are methods for imaging a nucleic acid target in a sample comprising contacting the sample comprising a plurality of distinct nucleic acid targets in situ with a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct nucleic acid target, and wherein each subpopulation of nucleic acid probes hybridizes to a distinct nucleic acid target; contacting the sample with a plurality of sandwich probes comprising two or more read sequences, wherein the sandwich probes hybridize to the converter sequences; contacting the sample with a plurality of readout probes comprising a fluorescent label, wherein the readout probes hybridize to the read sequences; imaging the readout probes hybridized to the read sequences; and, repeating steps contacting with readout probes and imaging in one or more sequential hybridization and imaging rounds until all positions in the valid codeword have been imaged providing imaged distinct nucleic acid targets.

Provided herein certain embodiments are methods for imaging RNA spatial organization in a sample comprising: a) contacting the sample comprising a plurality of distinct RNA species in situ with a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct RNA species, and wherein each subpopulation of nucleic acid probes hybridizes to a distinct RNA species; b) contacting the sample with a plurality of sandwich probes comprising two or more read sequences, wherein the sandwich probes hybridize to the converter sequences; c) contacting the sample with a plurality of readout probes comprising a fluorescent label, wherein the readout probes hybridize to the read sequences; d) imaging the readout probes hybridized to the read sequences; and, repeating steps c) and d) in one or more sequential hybridization and imaging rounds until all positions in the valid codeword have been imaged, providing an imaged codeword corresponding to each distinct RNA species in a spatial organization.

In certain embodiments provided herein are methods for generating in situ fiducial signals for image alignment in a sample, comprising contacting the sample comprising a plurality of distinct nucleic acid targets in situ with a plurality of primary nucleic acid probes each nucleic acid probe comprising (i) a target binding sequence and (ii) one or more converter sequences complementary to a sandwich probe, and wherein each subpopulation of nucleic acid probes hybridizes to a distinct nucleic acid target; contacting the sample with a plurality of sandwich probes comprising an in situ fiducial binding site wherein the sandwich probes hybridize to the converter sequences; contacting the sample with a plurality of fiducial probes comprising a fluorescent label, wherein the fiducial probes in situ fiducial binding site; and, imaging the readout probes hybridized to the read sequences; providing a reference signal for each nucleic acid target in the sample

Also provided herein are kits, in any configuration, comprising one or more of the primary nucleic acid probes, sandwich probes and readout probes for use in the methods of this disclosure.

### BRIEF DESCRIPTION OF THE DRAWINGS

FIG. 1 shows a schematic of representative primary nucleic acid probes of the present disclosure and use of the universal sequence.

FIG. 2 shows a schematic of representative primary nucleic acid probes of the present disclosure hybridized with sandwich probes.

FIG. 3 shows a schematic of representative primary nucleic acid probes of the present disclosure comprising a universal sequence, target binding sequence, converter sequences hybridized to the encoding sandwich probes as compared to a MERFISH encoding probe.

FIG. 4A shows a schematic of representative primary nucleic acid probes of the present disclosure comprising a fiducial binding site, target binding sequence, converter sequences hybridized to the sandwich probes and readout probes hybridized to the read sequences of the sandwich probes. The present methods use indirect binding of sandwich probes on primary nucleic acid probes to improve the signal to noise ratio for RNA imaging without creating an amplicon. The primary encoding probes comprises a target region that binds to the RNA transcripts, flanked by repeats of a converter sequence at the 5′ and 3′ end. The sandwich probes comprise a sequence that hybridizes to the converter sequences, and two readout sequences, a flanking region comprising 5T nucleotides, and an in situ fiducial binding site. In situ fiducial bindings sites are for image registration across different imaging rounds. Each readout probe is conjugated with two fluorescent dyes. At a minimal, two primary nucleic acid probes are required to form the Hamming weight 4 (HW4) codeword.

FIG. 4B shows an example of Hamming distance 4 (HD4) Hamming weight 4 (HW4) codeword.

FIG. 5 shows a representative workflow/protocol using nucleic acid probes of this disclosure with either fresh/frozen or FFPE treated tissue samples.

FIG. 6A shows a schematic of in situ fiducial probe binding. In embodiments, the sandwich probes comprise a 25nt in situ fiducial binding site, allowing the in situ fiducial probes to bind prior to the imaging step. See FIG. 5. The collective signal generated from all sandwich probes that bind to a sample can be used for image registration.

FIG. 6B shows in situ fiducial for image alignment across sequential imaging rounds in an example of in situ fiducial signal with 10 rounds of imaging. A mouse brain was processed and the in situ fiducial signal was displayed in each round for a single field of view.

FIG. 6C shows the principle of using in situ fiducials for fiducial warp between rounds.

FIG. 6D shows the principle of using in situ fiducial for global alignment between fields of views.

FIG. 6E demonstrates a successful decoding using in situ fiducial signal for image alignment. A mouse brain was stained with primary nucleic acid probes with or without in situ fiducial and processed for imaging. The image is then decoded for RNA detection. Detected counts per field of view is used to evaluate the performance of image alignment. The results show that in situ fiducial imaging performed better than image alignment using fiducial beads.

FIGS. 7A and B shows the nucleic acid probes and methods of this disclosure substantially improve RNA detection efficiencies in a wide range of samples from mouse (FIG. 7A) and human (FIG. 7B) as compared to methods in the art (e.g., MERFISH). To evaluate the performance of the nucleic acid probes and methods of this disclose, a wide range of samples from human (fresh frozen brain, FFPE breast cancer, FFPE lung cancer tissue microarray (TMA), FFPE breast cancer TMA) and mouse (fixed frozen spleen, heart, lung, small intestine, brain, kidney, liver and fresh frozen brain) were stained with different gene panel libraries following either MERFISH methods (black) or methods of the present disclosure (gray). RNA transcript counts per 100 μm² were used as a metric to evaluate sensitivity. Each condition has at least 3 samples (N=3). The present nucleic acid probes and methods demonstrate a significant increase in imaging of transcripts in a 100 μm² tissue section. See Example 2.

FIG. 8 shows the present nucleic acid probes and methods of this disclosure are consistent in technical replicates. Data within each testing group, mouse (FIG. 8A) and human (FIG. 8B), were used to perform correlation analysis. Person correlation coefficients from each analysis were used to plot. The correlation coefficients in all testing groups were close to 1.0.

FIG. 9 shows the nucleic acid probes and methods of this disclosure correlates well in a wide range of samples from mouse (FIG. 9A) and human (FIG. 9B) as compared to methods in the art (e.g., MERFISH). Data within each tissue were used to perform correlation analysis. Person correlation coefficients from each analysis were used to plot. The correlation coefficients in all testing groups were all above 0.8.

FIGS. 10A and B shows the nucleic acid probes and methods of this disclosure (FIG. 10B) substantially improve in situ gene expression profiling in human brain as compared to methods in the art (FIG. 10A) (e.g., MERFISH). Fresh frozen human brain was stained with a custom gene expression library and imaged. The imaged transcripts for OLIG2, MOG and SOX10 are shown along with a DAPI stain. A substantial increase of transcript counts for each gene were observed in FIG. 10B demonstrating the improvement of the present methods and nucleic acid probes.

FIGS. 11A and B shows the nucleic acid probes and methods of this disclosure (FIG. 11B) substantially improves dynamic range of gene expression in human brain cells as compared to methods in the art (FIG. 11A) (e.g., MERFISH). Fresh frozen human brain was stained with a custom library and the imaged data was used for downstream single cell analysis. FIGS. 11A and B show a histogram on of transcript/cell at the top of the figure and a spatial heat map of transcript/cell at the bottom of the figure.

FIG. 11C shows a violin plot of the dynamic range of RNA transcripts counts in six different brain cell types (glutamatergic neurons, GABAegeric neurons, oligodendrocytes, astrocytes, vascular cells, and microglia cells) using probes and methods of this disclosure (V2) as compared to methods in the art (V1).

FIG. 12A to 12F shows the nucleic acid probes and methods of this disclosure (FIGS. 12B and E) substantially improves imaging of low expression transcripts as compared to methods in the art (FIGS. 12A and D) (e.g., MERFISH). Fresh frozen human brain was stained with a custom library and the data was used for downstream single cell analysis. Two genes, GRIN1 (A-C) and CSFIR (D-F), were used to evaluate detection efficiency.

FIG. 12A shows a spatial distribution of gene GRIN1 and a spatial distribution of Leiden clusters that express GRIN1 with method of the art (e.g., MERFISH).

FIG. 12B shows a spatial distribution of gene GRIN1 and a spatial distribution of Leiden clusters that express GRIN1 with methods of this disclosure.

FIG. 12C shows quantification of GRIN expression in cells using MERFISH methods (left) and probes and methods of this disclosure (right).

FIG. 12D shows a spatial distribution of gene CSFIR and a spatial distribution of Leiden clusters that express CSFIR with method of the art (e.g., MERFISH).

FIG. 12E shows a spatial distribution of gene CSFIR and a spatial distribution of Leiden clusters that express CSFIR with methods of this disclosure.

FIG. 12F shows quantification of CSFIR expression in cells using MERFISH methods (left) and probes and methods of this disclosure (right).

FIG. 13A-C shows the nucleic acid probes and methods of this disclosure substantially improve RNA detection as compared to methods in the art (e.g., MERFISH). Fresh frozen human brain was stained with a custom 500 plex library. FIG. 13A shows the sensitivity comparison between MERFISH and methods of this disclosure using transcript counts per 100 μm² as a metric. FIG. 13B shows the correlation of in situ imaging data with bulk RNAseq, with person correlation coefficient used as a metric to indicate the accuracy of the measurement. FIG. 13C shows the correlation of MERFISH data with data generated from the methods of this disclosure.

FIG. 14A to 14E shows the nucleic acid probes and methods of this disclosure reduce run-to-run variation and cell dropout rate for downstream analysis as compared to methods in the art (e.g., MERFISH). Fresh frozen mouse and human brain was stained with a custom 500 plex library and samples imaged on the MERSCOPE instrument. FIG. 14A shows the sensitivity comparison in mouse brain-transcript counts per 100 μm² (top) and histogram showing the transcripts/cell (bottom).

FIG. 14B shows the sensitivity comparison in human brain-transcript counts per 100 μm² (top) and histogram showing the transcripts/cell (bottom).

FIG. 14C shows the Uniform Manifold Approximation and Projection (UMAP) displaying the different cell types identified in human brain.

FIG. 14D shows the spatial distribution of cell types identified with MERFISH methods (top 25,755 cells) and methods of this disclosure (bottom; 42,750 cells).

FIG. 14E shows proportion of cells identified in each cluster with MERFISH on bottom (black) and methods of this disclosure on top (gray). A higher fraction of cells was identified with methods of this disclosure compared to MERFISH methods.

FIG. 15A to 15D shows the nucleic acid probes and methods of this disclosure substantially improve sensitivity for RNA detection in FFPE human breast cancer samples as compared to methods in the art (e.g., MERFISH). FFPE human breast cancer TMA samples were stained with custom 960 plex libraries and imaged. FIG. 15A shows DAPI stain of the FFPE human breast cancer tissue microarray (TMA). FIG. 15B shows a histogram of the transcript/cell with MERFISH and methods of this disclosure in each core of the TMA from different regions (e.g., R1) of the TMA core. FIG. 15C shows UMAP displaying the different cell types identified in human breast cancer TMA. FIG. 15D shows spatial distribution of cell types identified in TMA from five different regions (R1 to R5), with the top row using method of the art (e.g., MERFISH), and the bottom row using methods of this disclosure.

FIG. 16A to 16E shows the nucleic acid probes and methods of this disclosure substantially improve single cell analysis in FFPE human breast cancer samples as compared to methods in the art (e.g., MERFISH). FFPE human breast cancer TMA samples were stained with custom 960 plex libraries and imaged and the data was then used for single cell analysis. FIG. 16A shows a correlation of cell clusters identified with MERFISH (Y-axis) and methods of this disclosure (X axis). FIG. 16B shows a correlation of gene expression between MERFISH (X-axis) and methods of this disclosure in vascular cells (Y axis). The high correlation coefficient indicates that cell types identified by the two methods correlate well, despite the sensitivity differences. FIG. 16C shows correlation with single cell RNA sequencing by MERFISH (V1) and methods of this disclosure (V2), with present methods improving the correlation coefficient. FIG. 16D shows a violin plot of the dynamic range of RNA transcript counts in select identified cell types in the human breast cancer with MERFISH methods on the left of each plot and the methods of this disclosure on the right of each plot. FIG. 16E shows spatial distribution of T cells (Left) and CD3E gene expression (Right) in human breast cancer core with MERFISH (top) and methods of this disclosure (bottom); and quantification of CD3E counts within T cells with MERFISH (left) and methods of this disclosure (right).

FIG. 17A to 17C shows the nucleic acid probes and methods of this disclosure provide better characterization of spatial interactions in human breast cancer as compared to methods in the art (e.g., MERFISH). FFPE human breast cancer TMA samples were stained with custom 960 plex libraries, images and the data was then used for single cell analysis with the top row of images generated using MERFISH methods and the bottom row with methods of this disclosure. FIG. 17A shows the spatial distribution of major cell types in the human breast cancer core (top: method of the art (e.g., MERFISH), bottom, methods of this disclosure). FIG. 17B shows the spatial enrichment analysis of major cell types (top: method of the art (e.g., MERFISH), bottom, methods of this disclosure). FIG. 17C shows the spatial distribution of B and T cells in the core wherein the spatial enrichment of T and B cells were detected with the methods of this disclosure but not MERFISH methods (top: method of the art (e.g., MERFISH), bottom, methods of this disclosure).

FIG. 18A to 18D show the nucleic acid probes and methods of this disclosure substantially improve sensitivity for RNA detection in FFPE human lung cancer samples as compared to methods in the art (e.g., MERFISH). FFPE human lung cancer TMA samples were stained with custom 960 plex libraries and imaged; the image with an X was not used for analysis. FIG. 18A shows DAPI stain of the FFPE human lung cancer TMA. FIG. 18B shows a histogram of the transcript/cell with MERFISH and methods of this disclosure in each core of the TMA. FIG. 18C shows UMAP displaying the different cell types identified in human breast cancer TMA. FIG. 18D shows the spatial distribution of cell types identified in TMA with MERFISH (left) and methods of this disclosure (right).

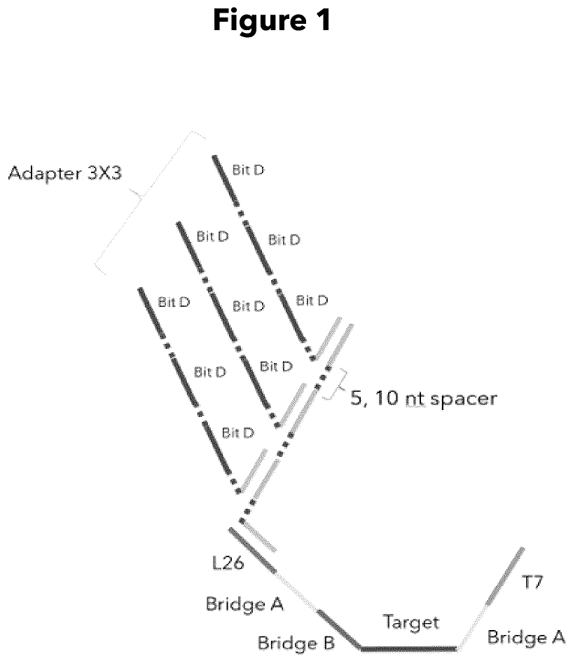

### DETAILED DESCRIPTION OF THE DISCLOSURE

### A. Overview

The present disclosure generally relates to primary nucleic acid probes for use in a multiplex assay for in situ nucleic acid detection and imaging. The primary nucleic acid probes of this disclosure are designed and generated to hybridize with a cellular nucleic acid target(s) (comprise a target binding sequence) and also contain converter sequences (configured to hybridize to sandwich probes) wherein each unique converter sequence is assigned to two or more positions in a valid codeword assigned to a distinct nucleic acid target. Those converter sequences are then read out in a serious of hybridization steps using a sandwich probe and readout probes, wherein the sandwich probes comprise one or more read sequence and optionally an in situ fiducial binding site. These primary nucleic acid probes provide an improvement over current MERFISH encoding probes and are structurally and functionally distinct from those probes. The improvement including signal uniformity, sensitivity, accuracy and brightness. MERFISH encoding probes, and their decoding schemes, are disclosed in U.S. Pat. No. 11,098,303 (the '303 patent) or U.S. Pat. No. 10,240,146, each incorporated herein by reference in its entirety. Those patents disclose the use of error-robust in situ hybridization nucleic acid probes (“encoding probes”) that reduce misidentification of nucleic acid targets in a sample wherein probe pools for distinct targets encode a N-bit binary code. Those nucleic acid probes comprise a target sequence and one or more read sequences wherein the read sequence corresponds to a single bit value of the N-bit code, which is imaged in sequential hybridization rounds using fluorescent readout probes that bind to the read sequences of the nucleic acid probes.

We herein provide improved (“primary nucleic acid probes”) and sandwich probes configured to hybridize to the converter sequences of the primary nucleic acid probes. We observed, when sequencing those MERFISH encoding probes, which comprise a target binding sequence and one or more readout sequences, some bias in the output probe abundance after our internal amplification process—it appeared that some readout sequences were largely depleted during the amplification process, depleting the relative abundance of some bits (i.e., read sequences) relative to others. To improve encoding probe design for improved signal uniformity, sensitivity, accuracy and brightness we introduced a sandwich probe between the primary nucleic acid probe and the readout probe with a 2:1 design where every possible pair of bits or positions in the codeword was assigned a unique converter sequence. The primary nucleic acid probes were constructed with the target binding regions associated with the bit pair sequences via the converter sequence. Since each barcode used 4 ‘1’ bits, each barcode was associated with six bit pair sequences. Then, the corresponding nucleic acid probes were constructed to include each of these per bit pair sequences. Each primary nucleic acid probe was designed to only have one per bit pair sequence (two copies of it) to avoid having more than 2 readout sequences associated with a given primary nucleic acid probe. With this design, if some of the per bit pair adapter sequences are disfavored by our probe amplification process, two other per bit pair sequences will still be associated with either bit so the bit will still be present at reasonable abundance in the final probe sets.

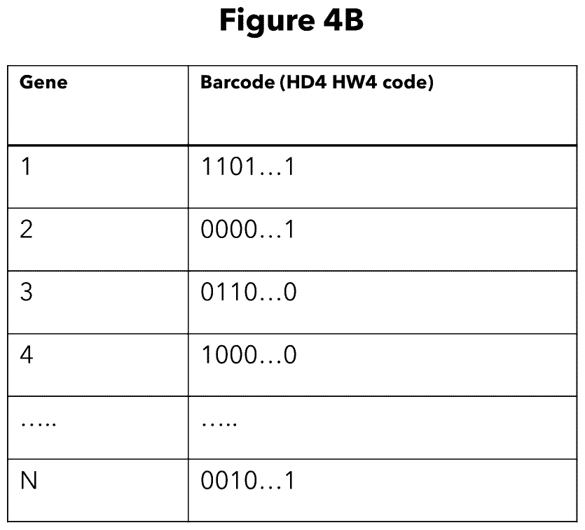

In embodiments provided herein is a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct nucleic acid target, and wherein each subpopulation of nucleic acid probes hybridizes to a distinct nucleic acid target. In embodiments, the sandwich probe comprises two or more read sequences, wherein the sandwich probes hybridize to the converter sequences. In embodiments, every nucleic acid target (e.g., RNA or DNA) is assigned two unique converter sequences, one half of the primary encoding probes are generated comprising converter sequence 1, and the second half of the primary encoding probes are generated comprising converter sequence 2. In this way, when used in the present methods converter sequence 1 converts to bit 1, 2, and converter sequence 2 converts to bit 3 and 4 of a valid codeword. See FIG. 4.

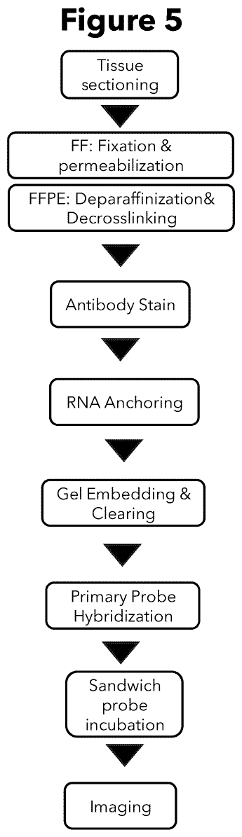

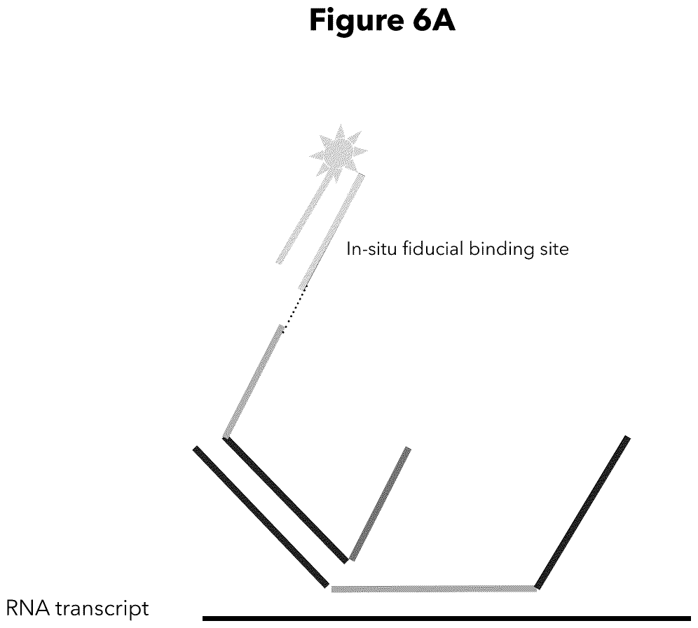

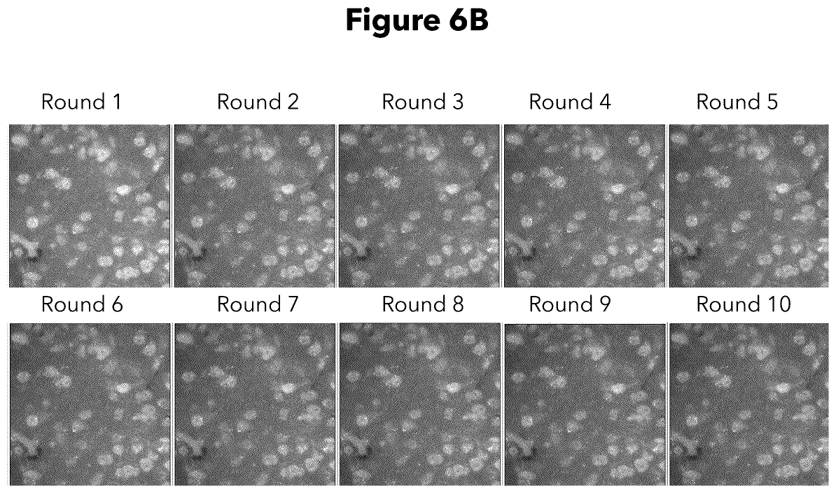

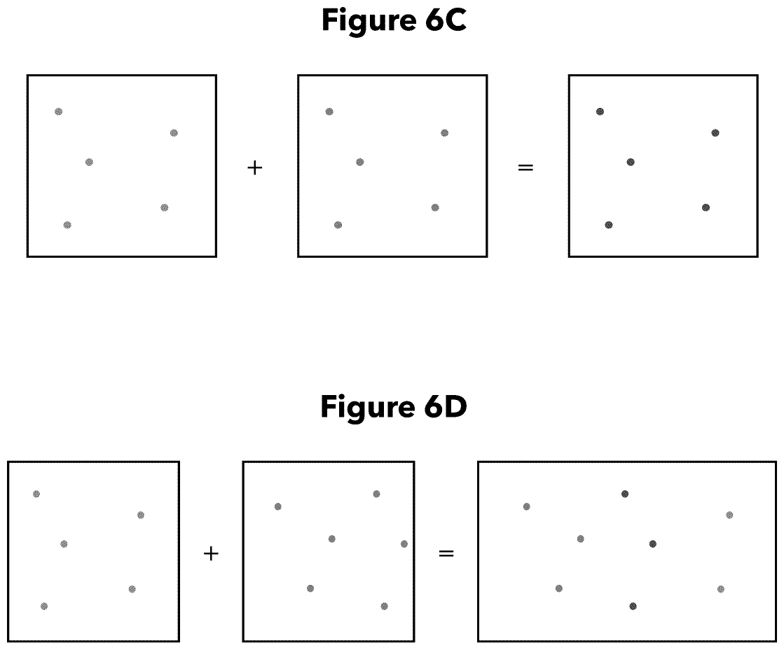

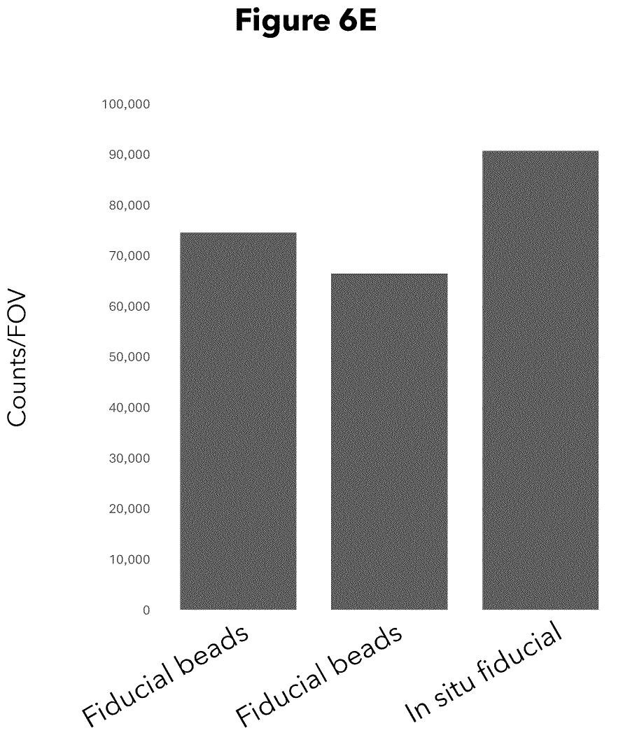

In embodiments provided herein are methods for imaging a nucleic acid target in a sample, comprising contacting the sample comprising a plurality of distinct nucleic acid targets in situ with a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct nucleic acid target, and wherein each subpopulation of nucleic acid probes hybridizes to a distinct nucleic acid target. The sample is then contacted with a plurality of sandwich probes comprising two or more read sequences, wherein the sandwich probes hybridize to the converter sequences. The sample is further contacted with a plurality of readout probes comprising a fluorescent label, wherein the readout probes hybridize to the read sequences and then imaged. See FIG. 5.

By way of example only, for a 30-bit measurement (meaning the valid codeword comprises 30 positions), there are 435 possible per bit pair combinations. Each of these bit pairs is assigned a unique converter sequence—the per bit pair sequence. To construct the primary nucleic acid probes and sandwich probes for a gene, the gene is assigned a barcode with four ‘1’ bits, then the bit pair sequences for the 6 possible combinations of ‘1’ bits ((1,2), (1,3), (1,4), (2,3), (2,4), (3,4)) are used to construct the primary nucleic acid and encoding probes. Specifically, there are six different per bit pair sequences that are used to construct the primary nucleic acid probes for that gene and none of these sequences correspond directly to a readout sequences. Those are the “unique converter sequences” of the primary nucleic acid probes and the sequence that is complementary and hybridizes to the sandwich probe. After the primary nucleic acid probes with the per bit pair converter sequences (“unique converter sequence”) are hybridized, probes constructed from the per bit pair converter sequences and the corresponding bit sequences (“sandwich probes”) are hybridized to convert the per bit pair sequences into the two corresponding bit pair sequences. These corresponding bit sequences (“read sequences”) can then be read out through sequential hybridization as in a standard MERFISH measurement using readout probes.

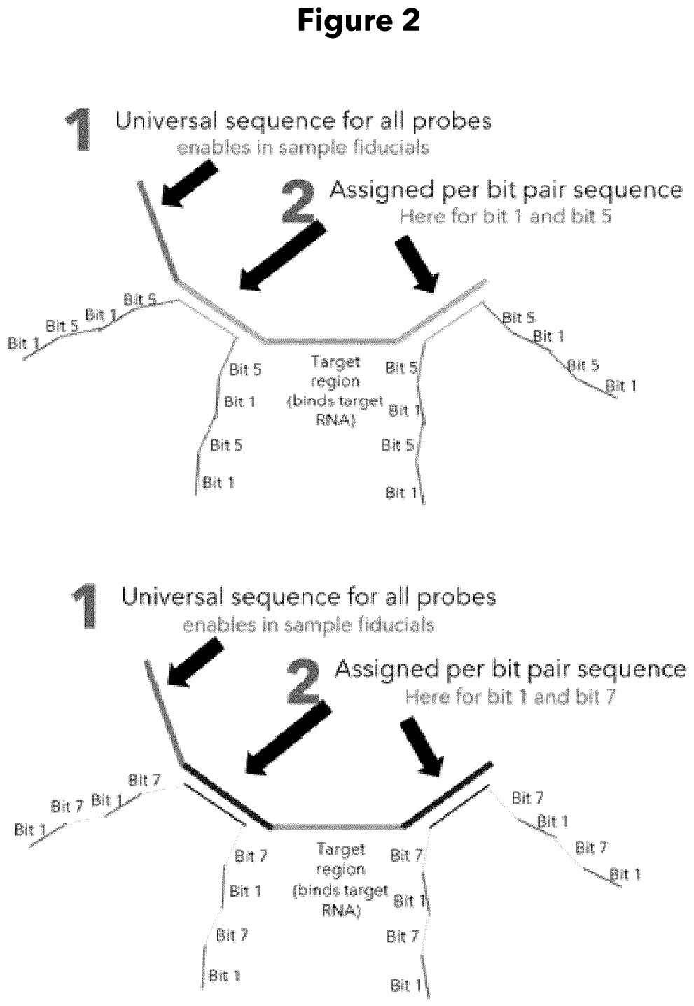

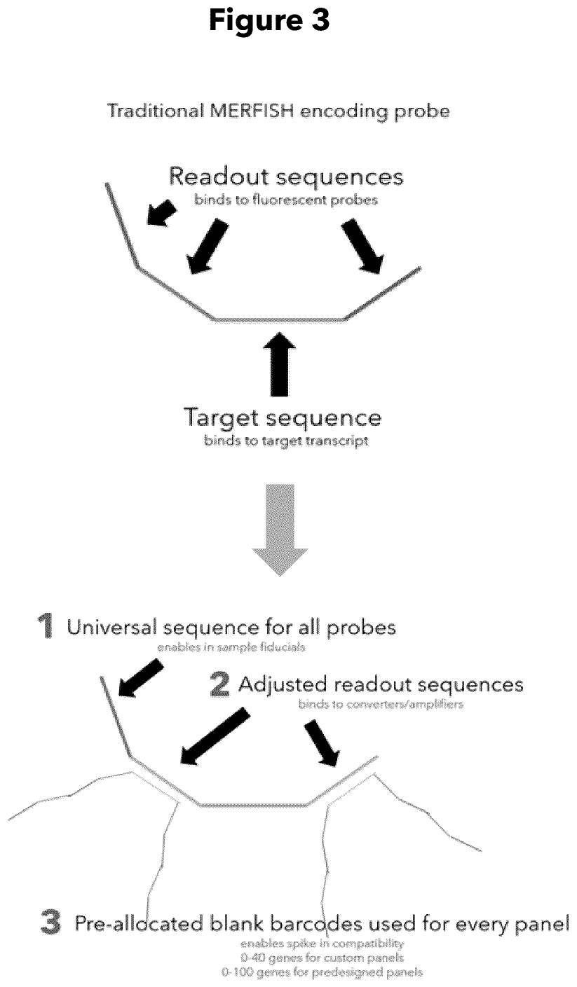

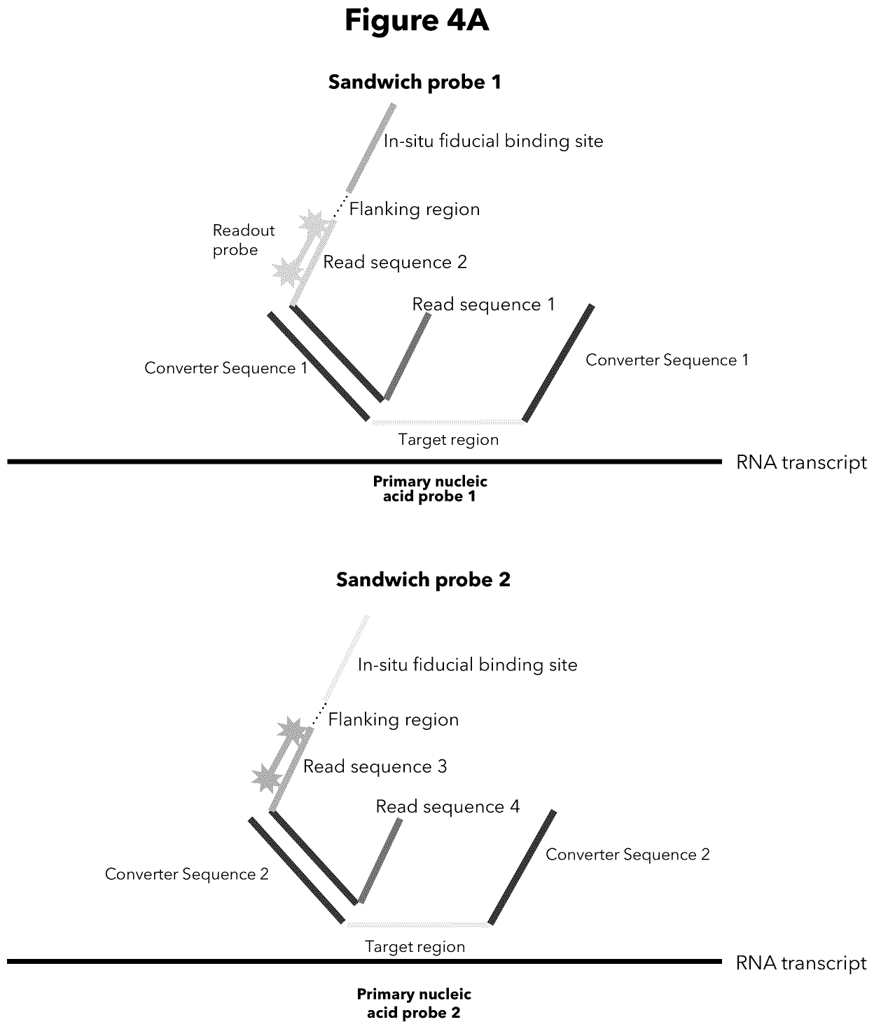

FIGS. 2, 3 and 4 illustrates this per bit pair scheme for two bit pairs (bit 1, bit 5) and (bit 1, bit 7), demonstrating how the per bit pair sequence on the primary nucleic acid probe is used to convert to the two corresponding bit sequences through the adapter (sandwich encoding probe) that hybridizes to the per bit pair sequence on the primary nucleic acid probe. As an example, a gene that is assigned a barcode with ‘1’ bits of bit 1, bit 5, bit 7, and bit 11 would use these two bit pair sequences on its encoding probes, in addition to the bit pair sequences for (bit1, bit 11), (bit 5, bit7), (bit 5, bit 11), and (bit 7, bit 11).

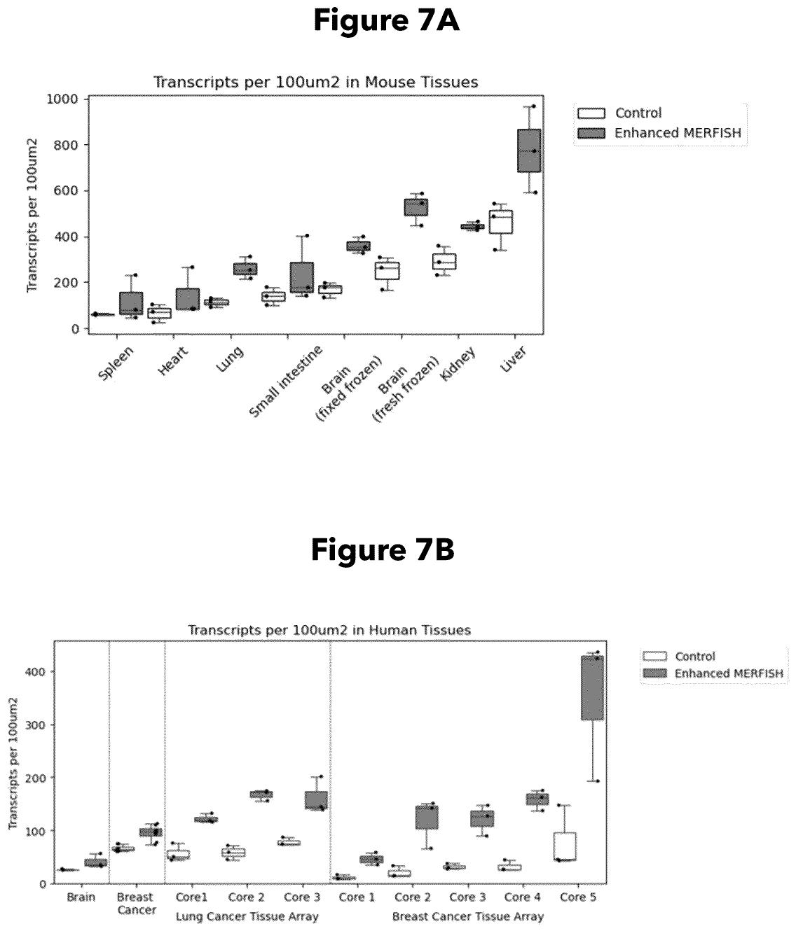

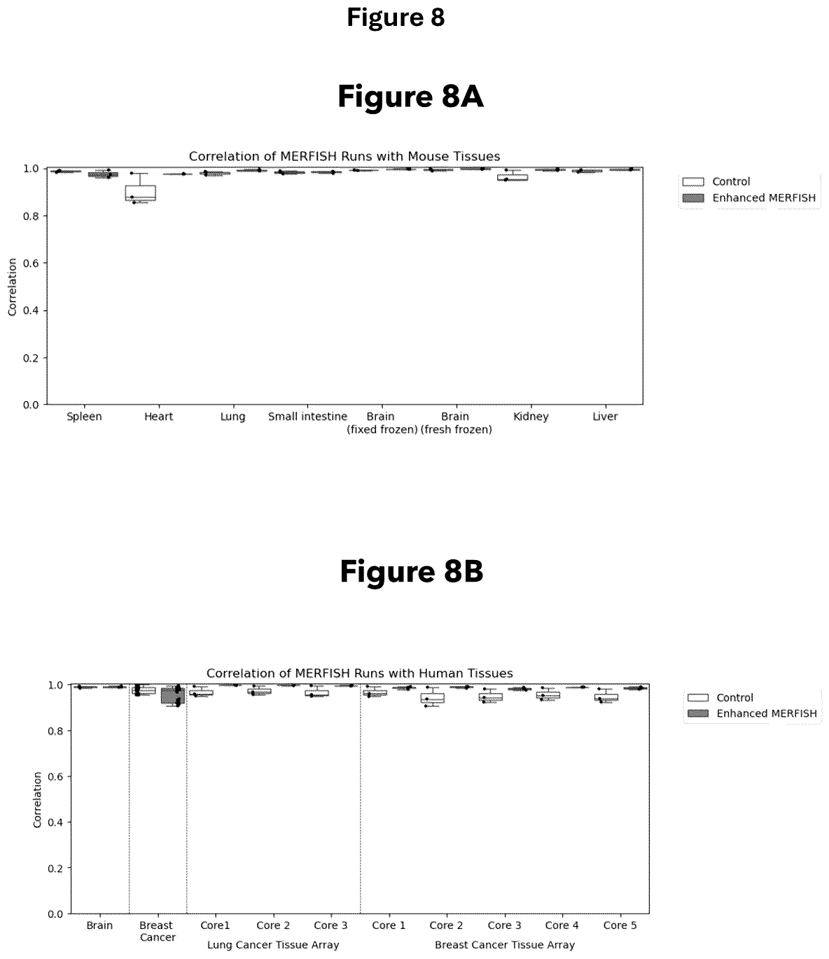

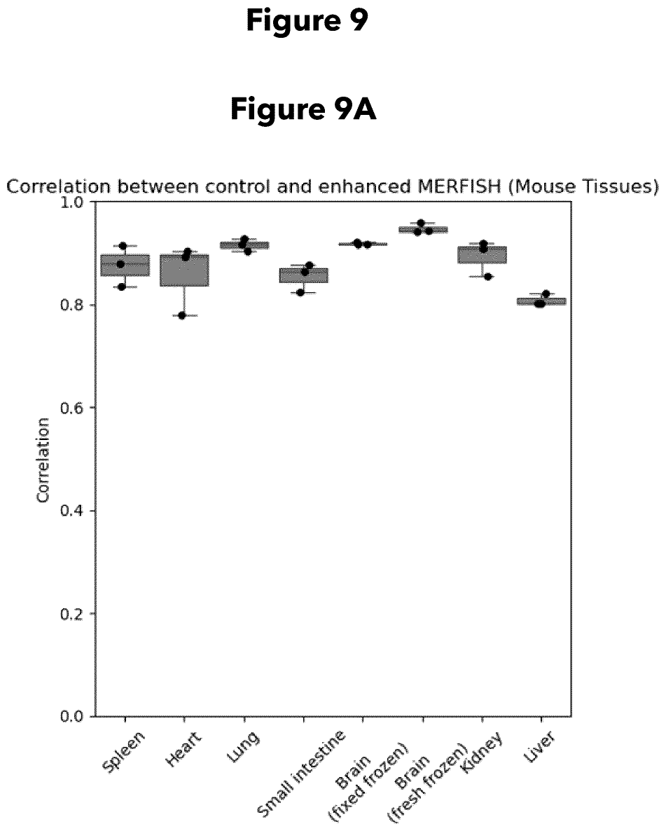

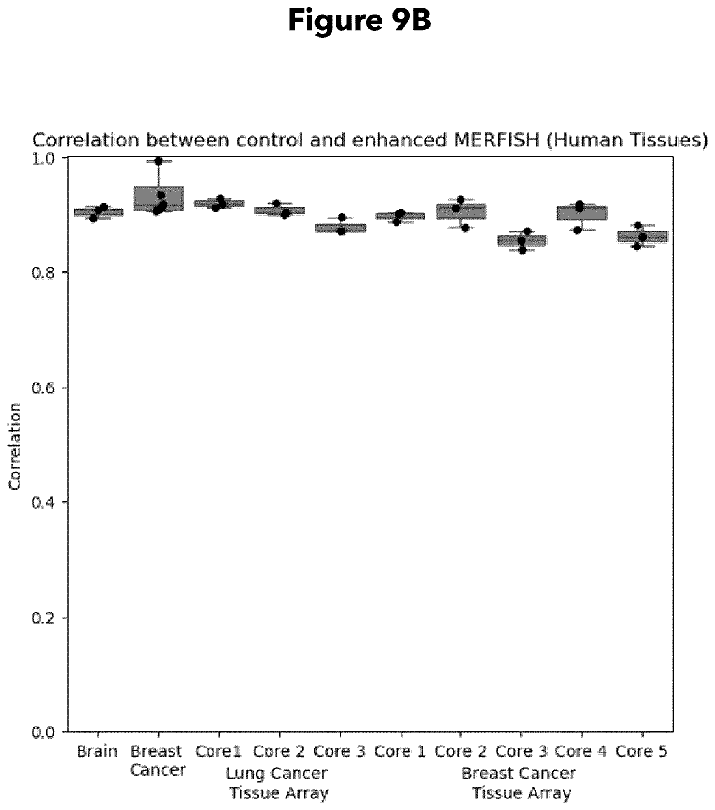

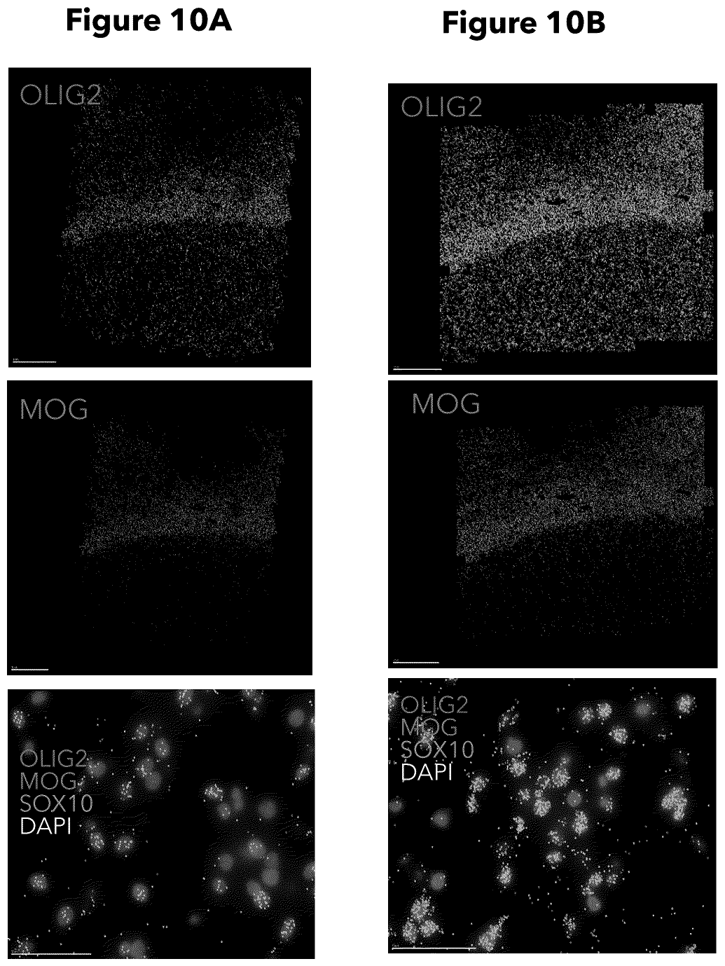

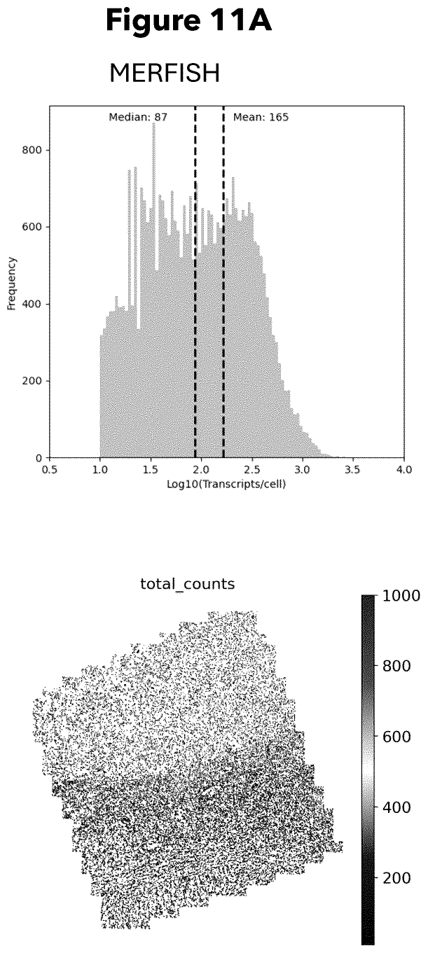

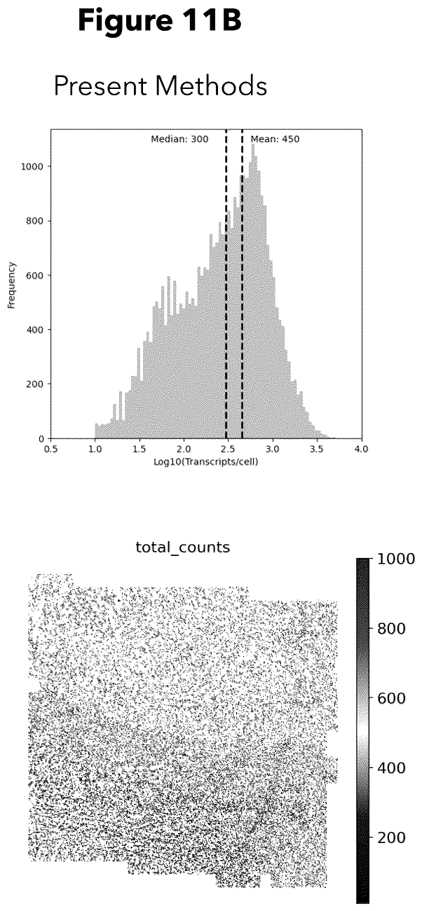

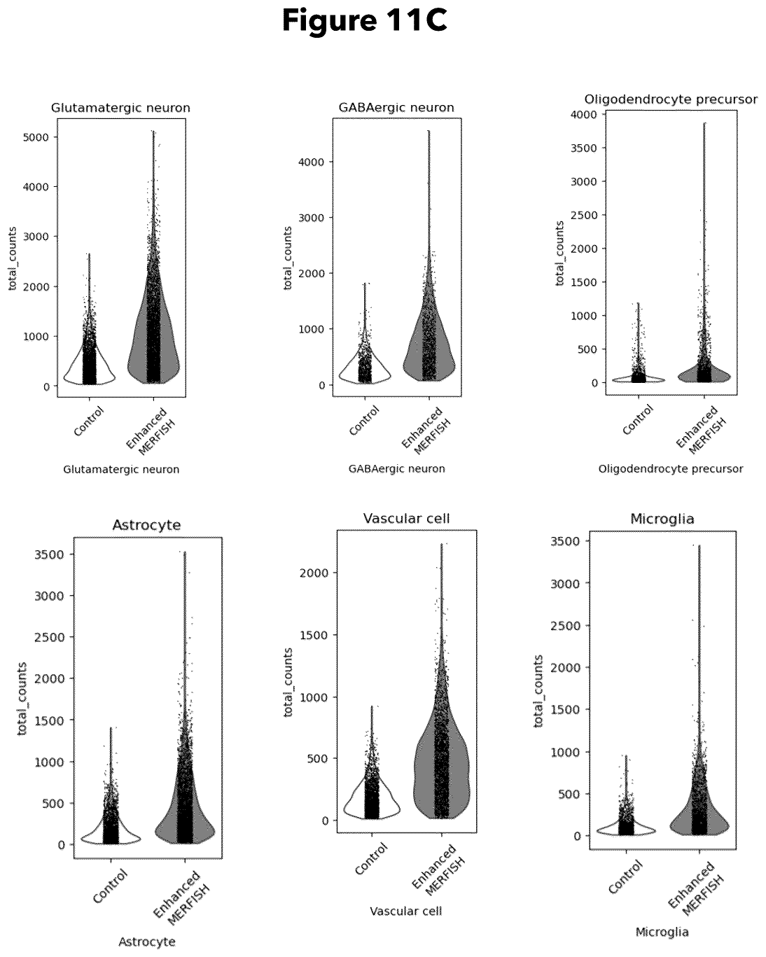

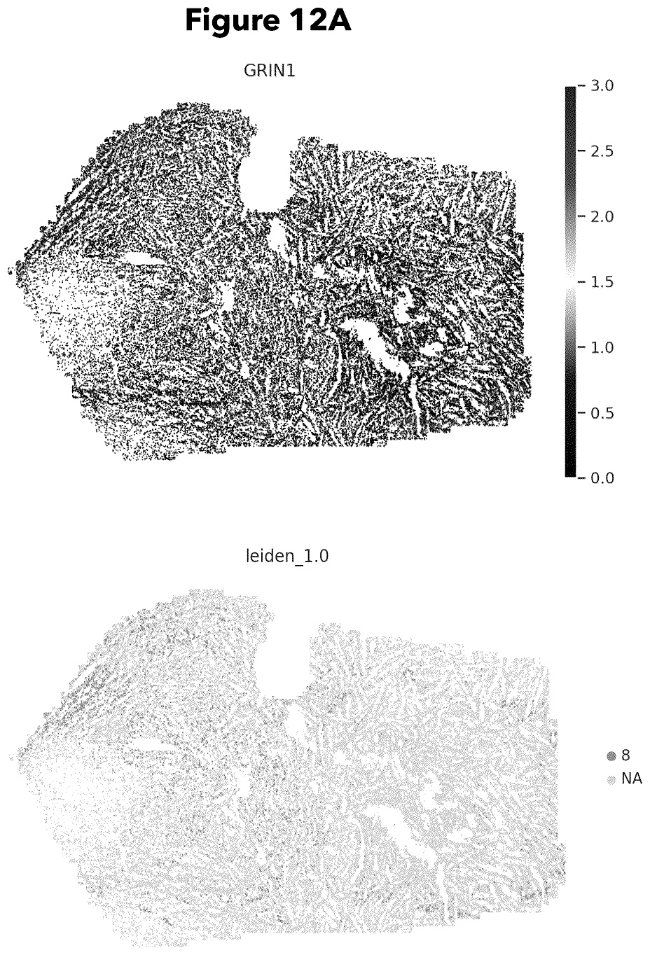

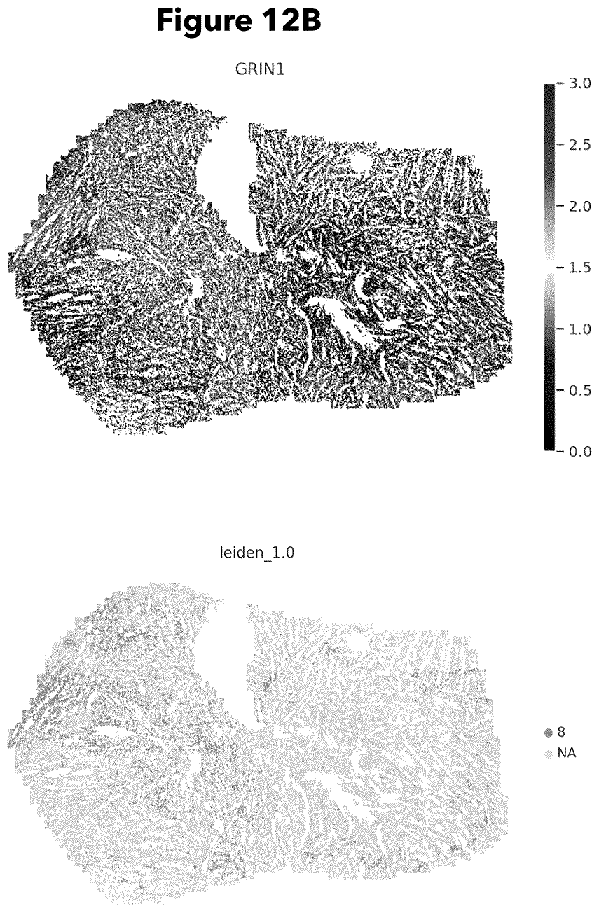

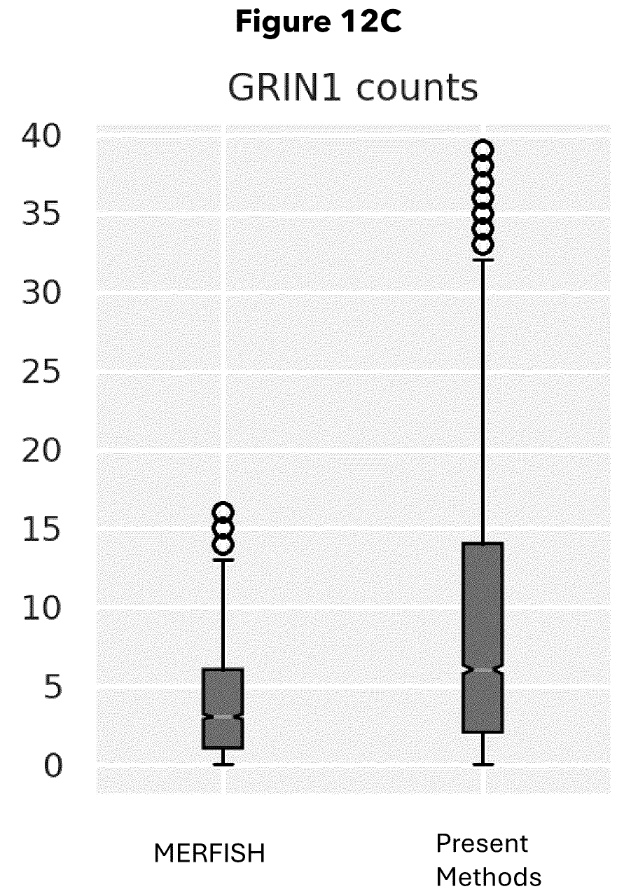

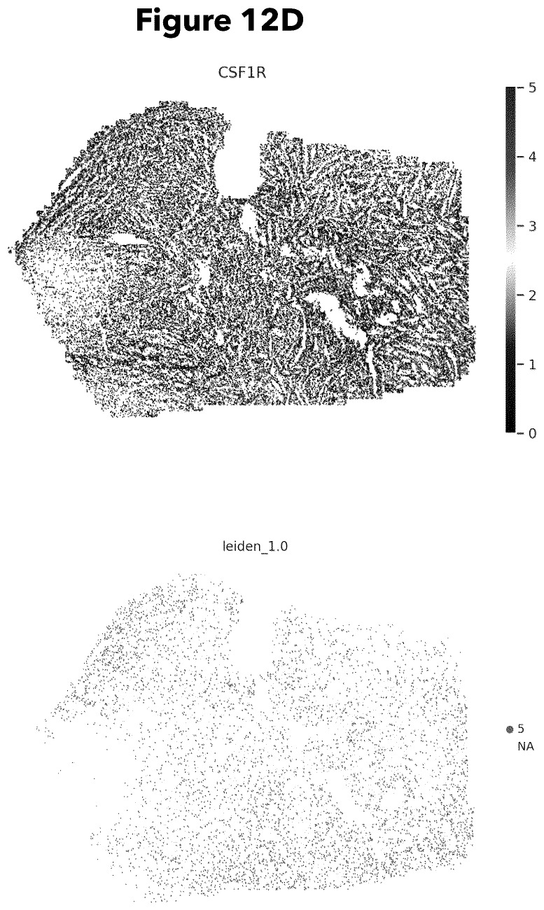

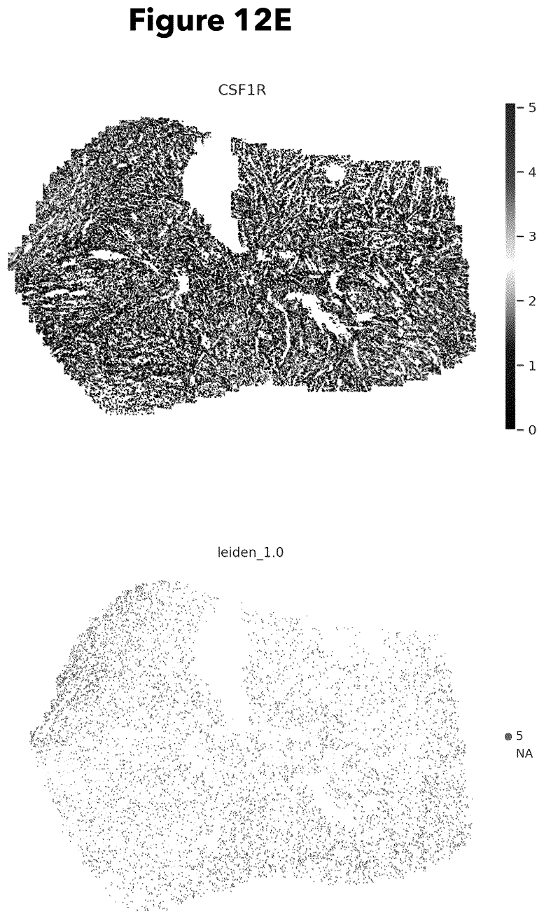

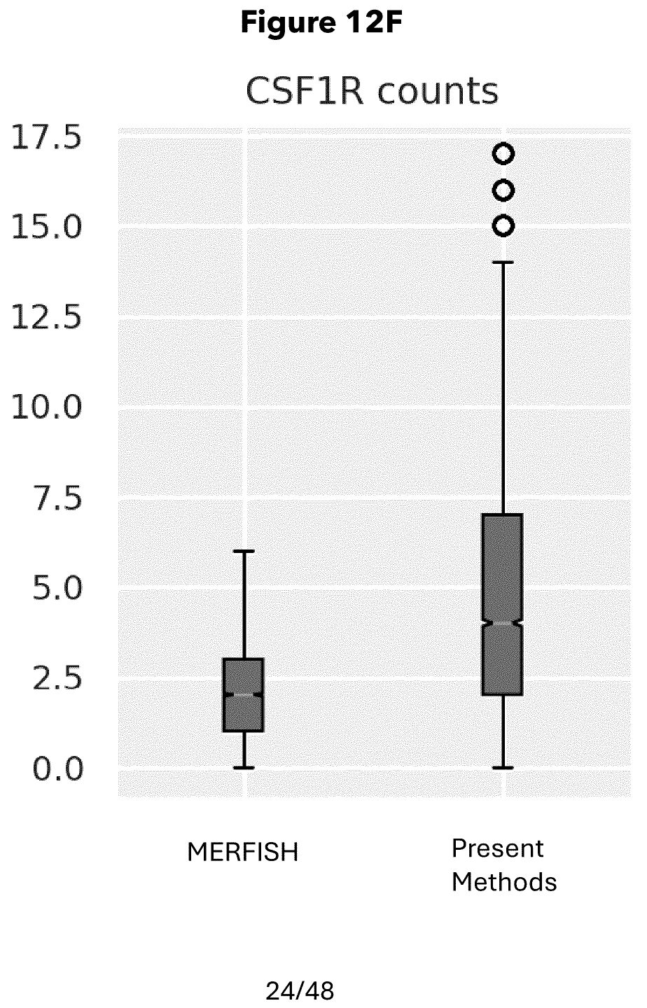

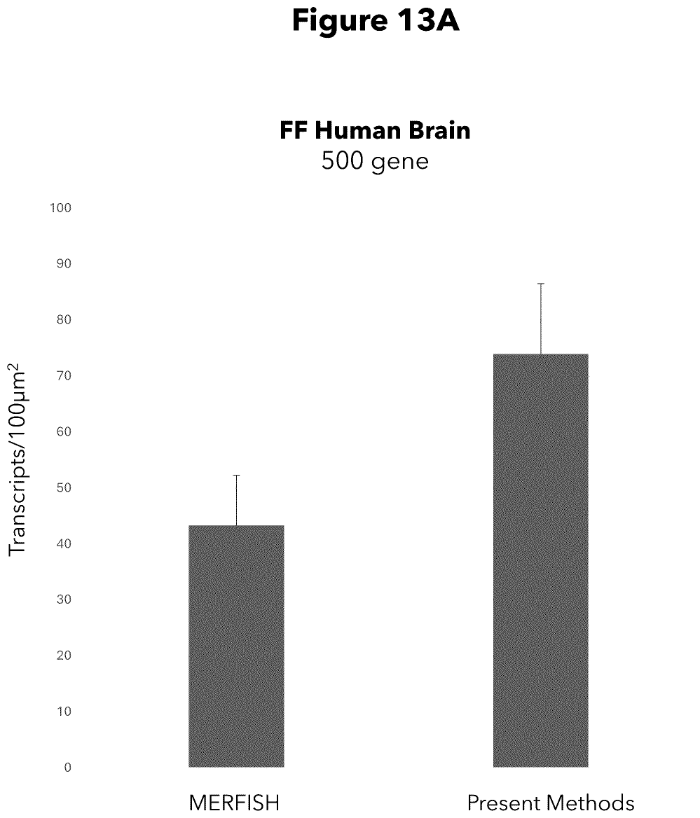

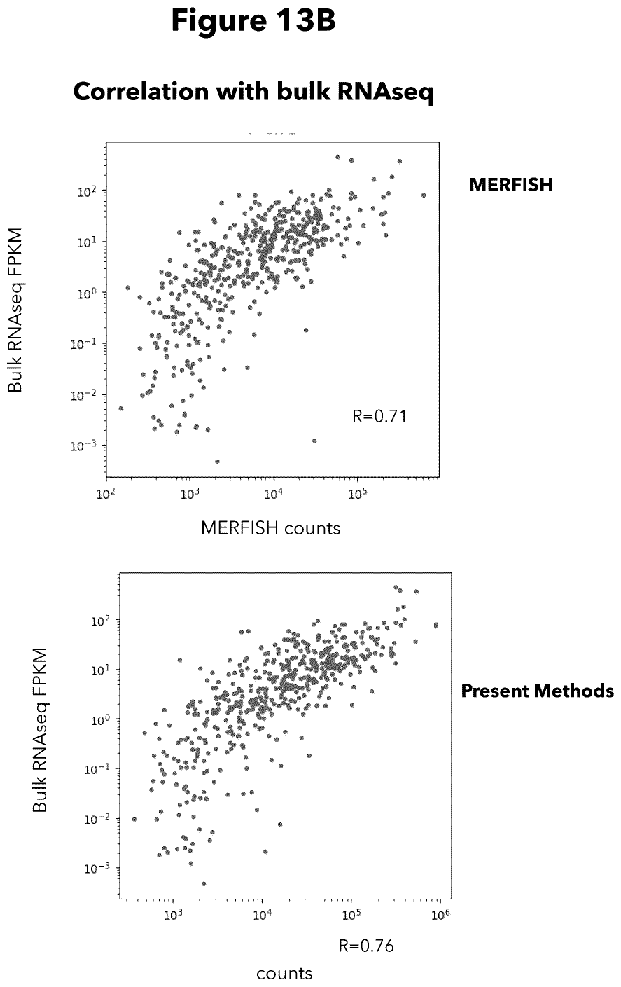

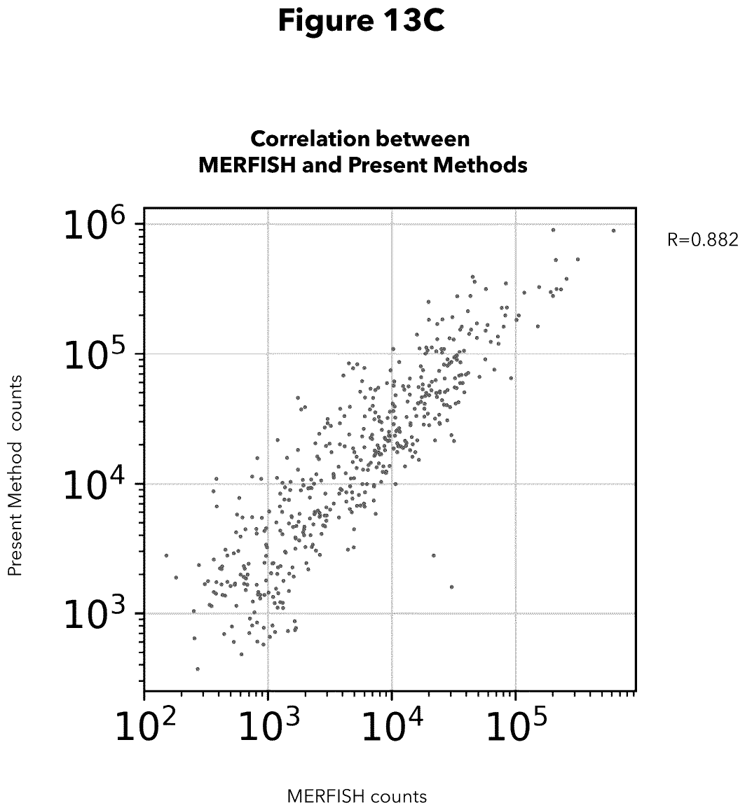

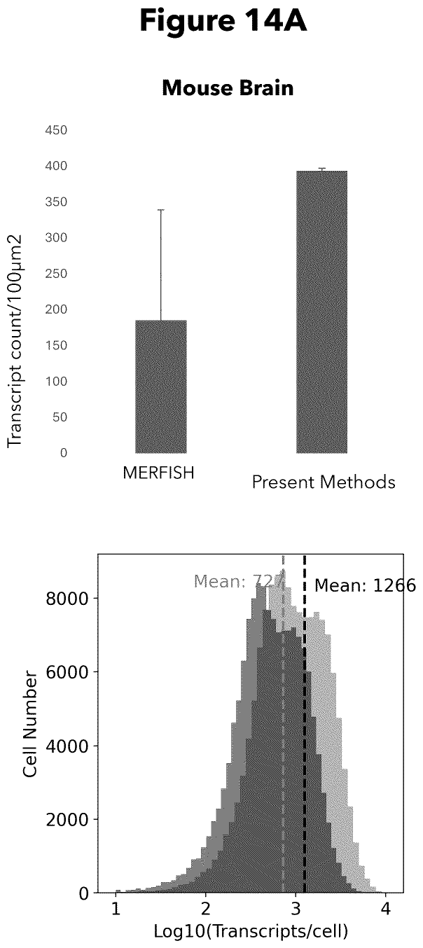

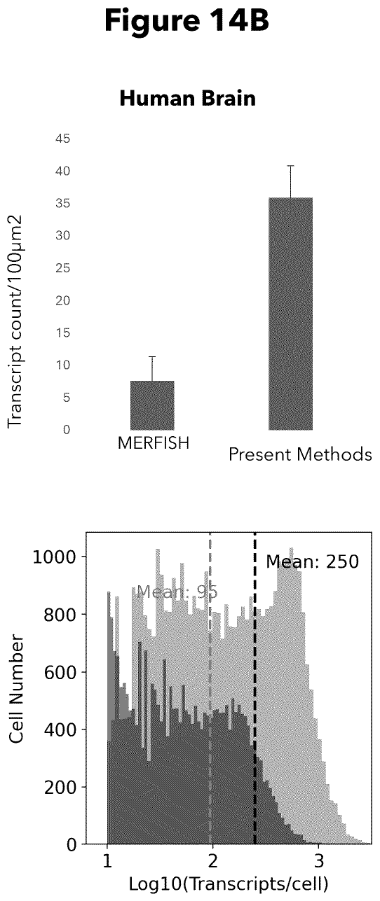

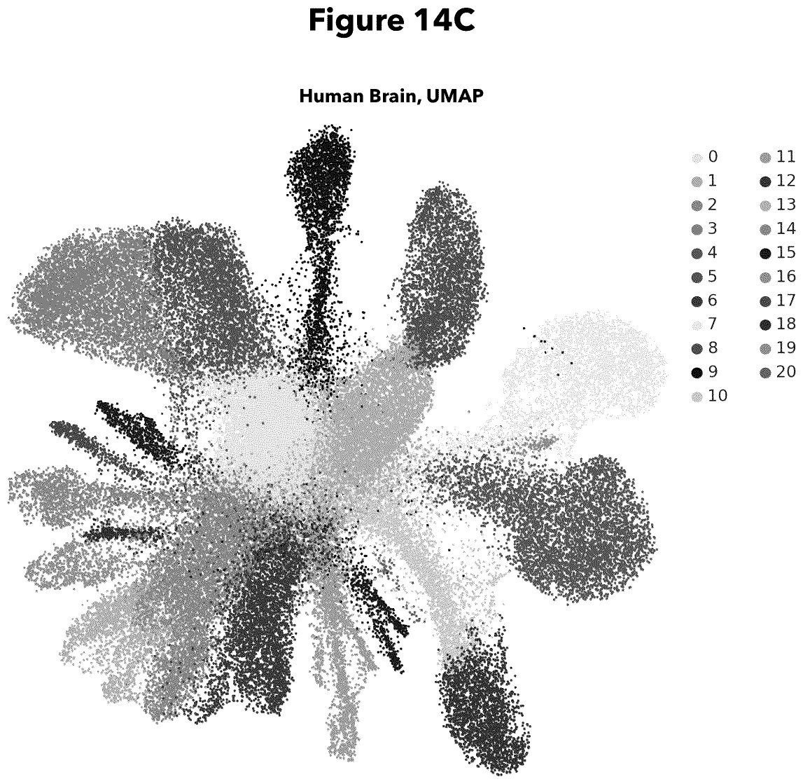

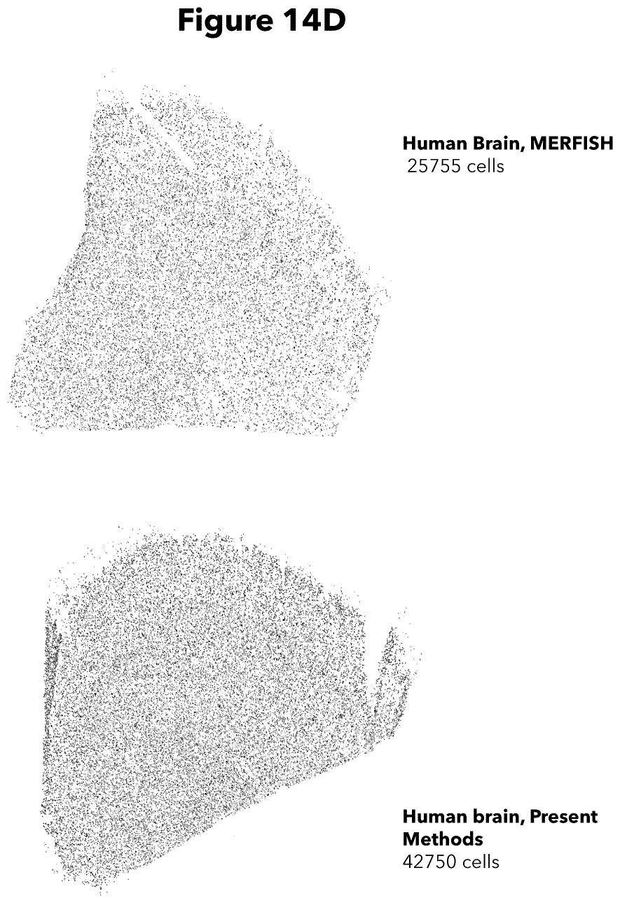

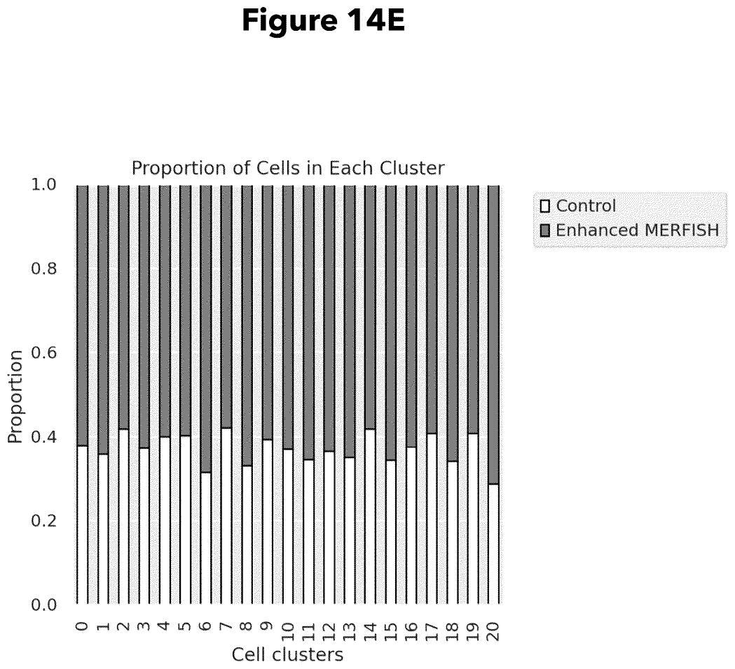

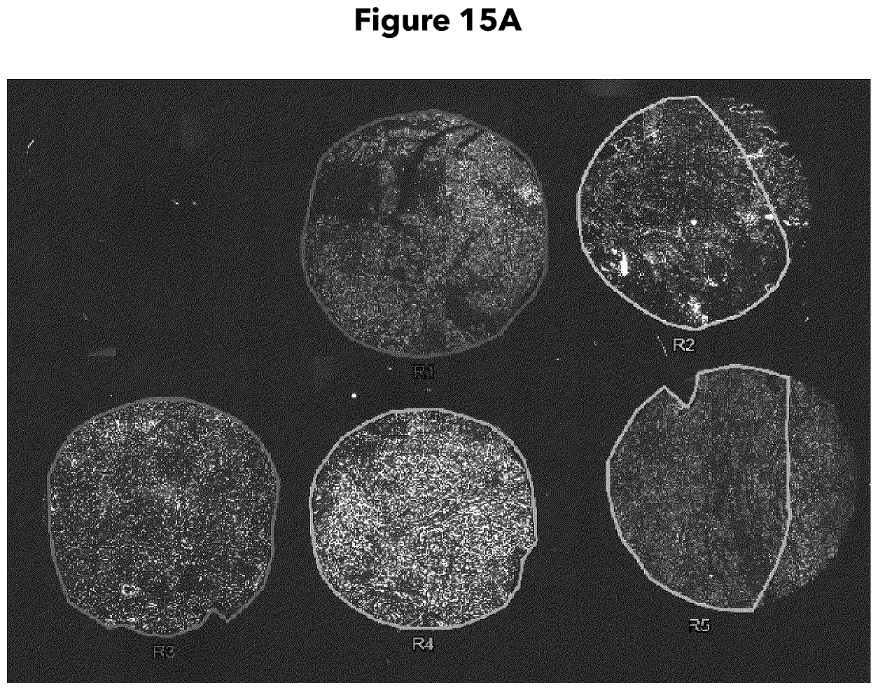

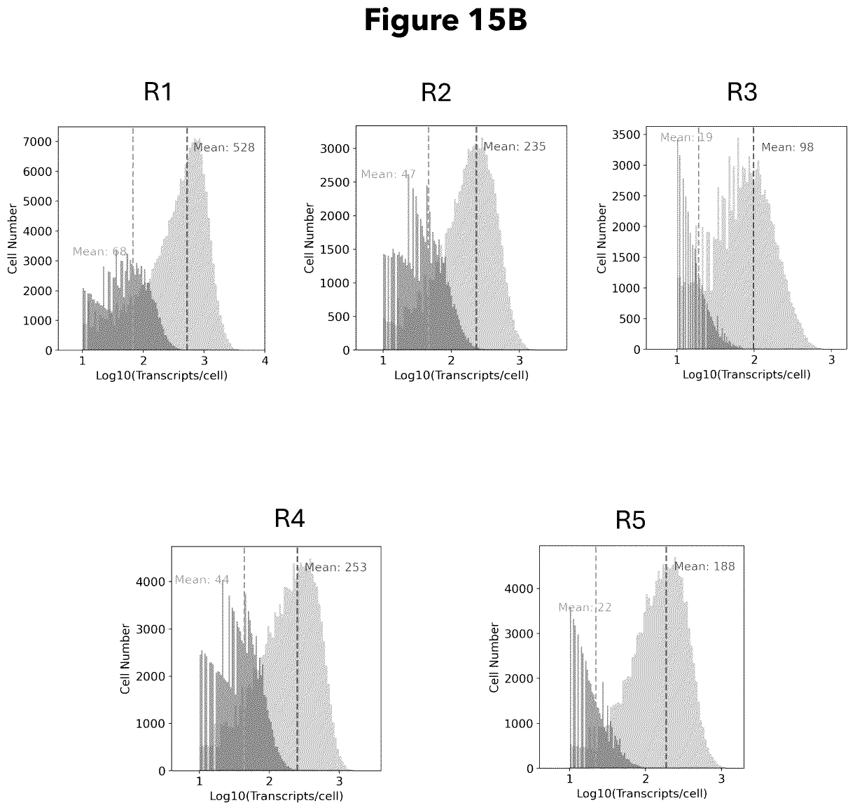

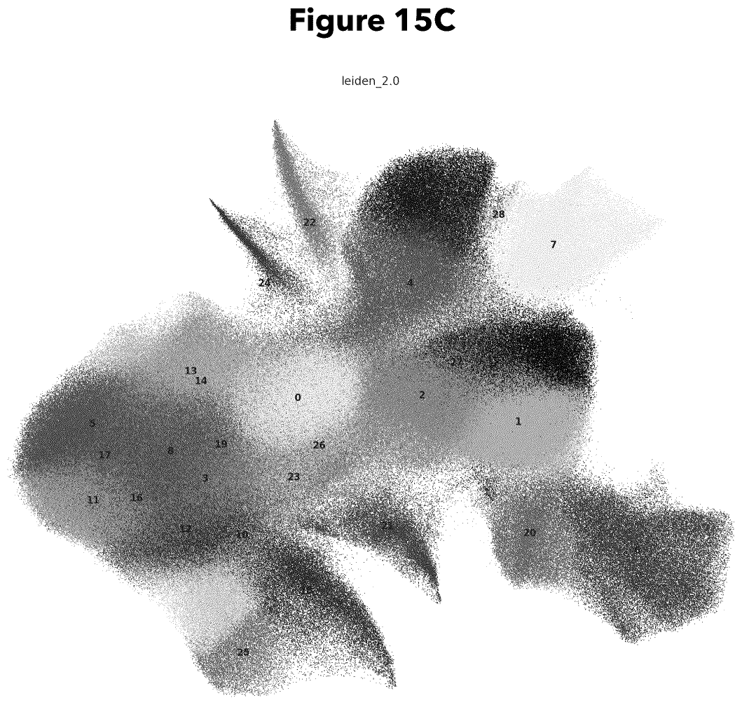

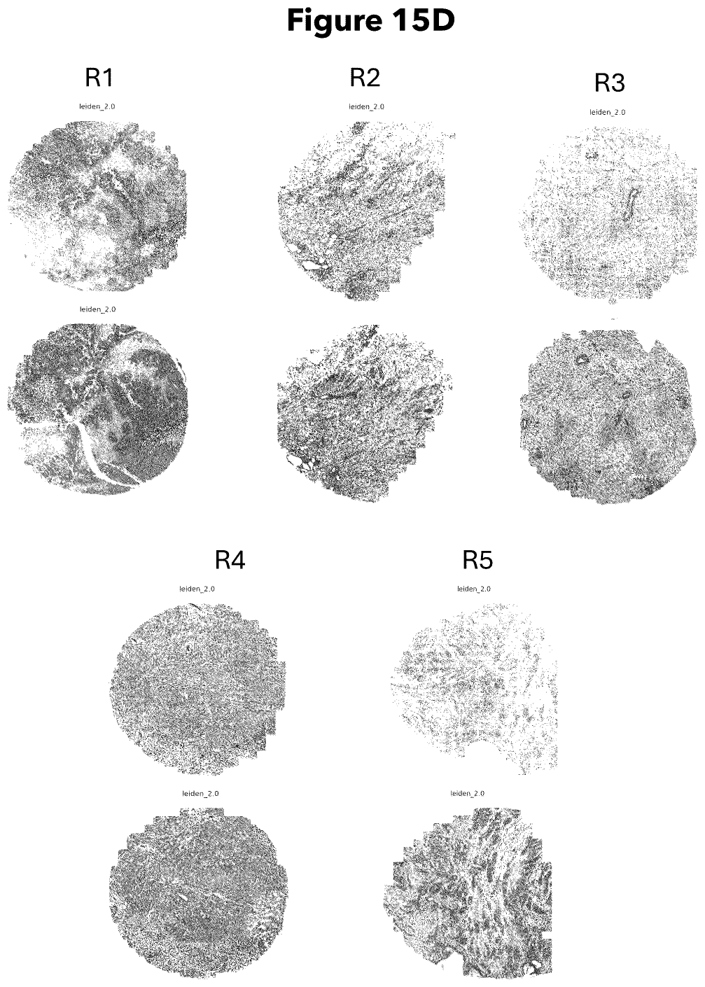

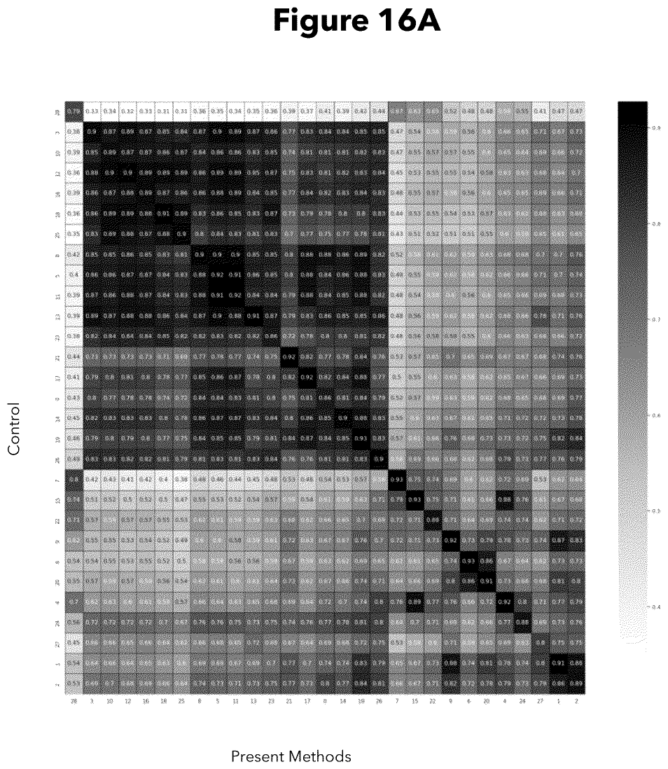

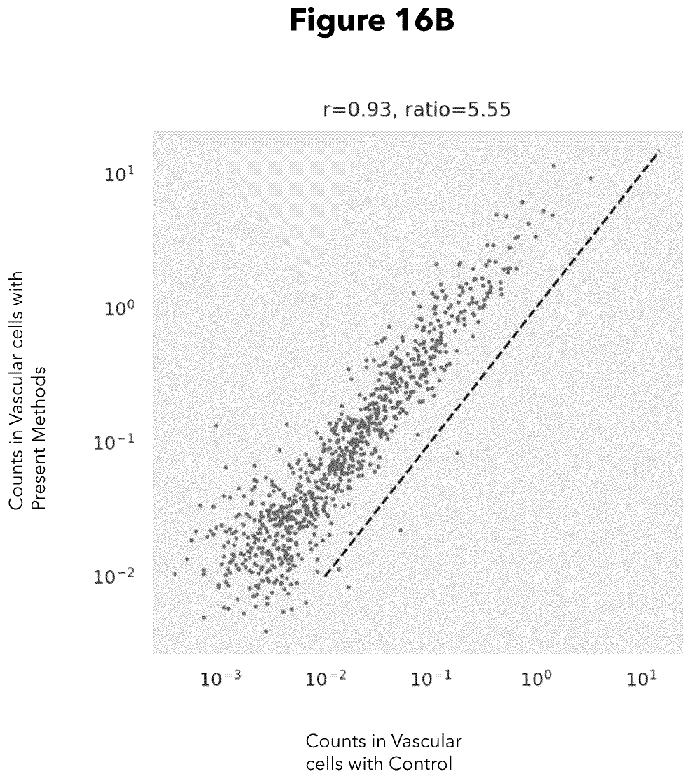

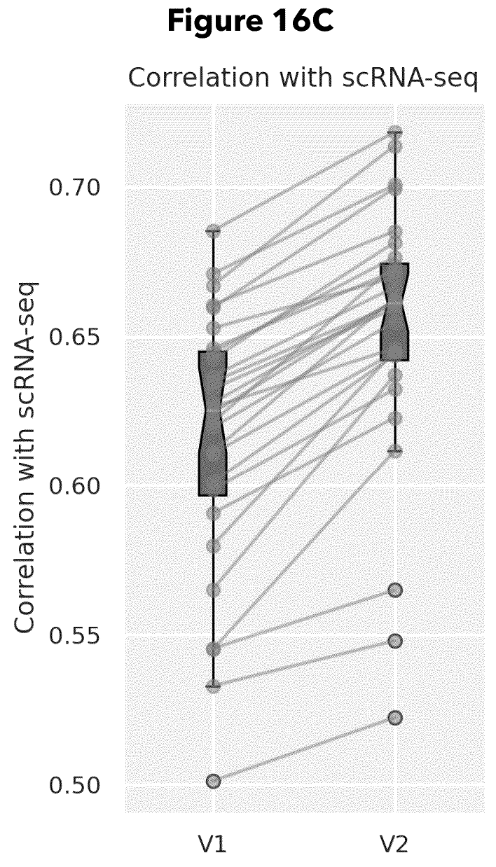

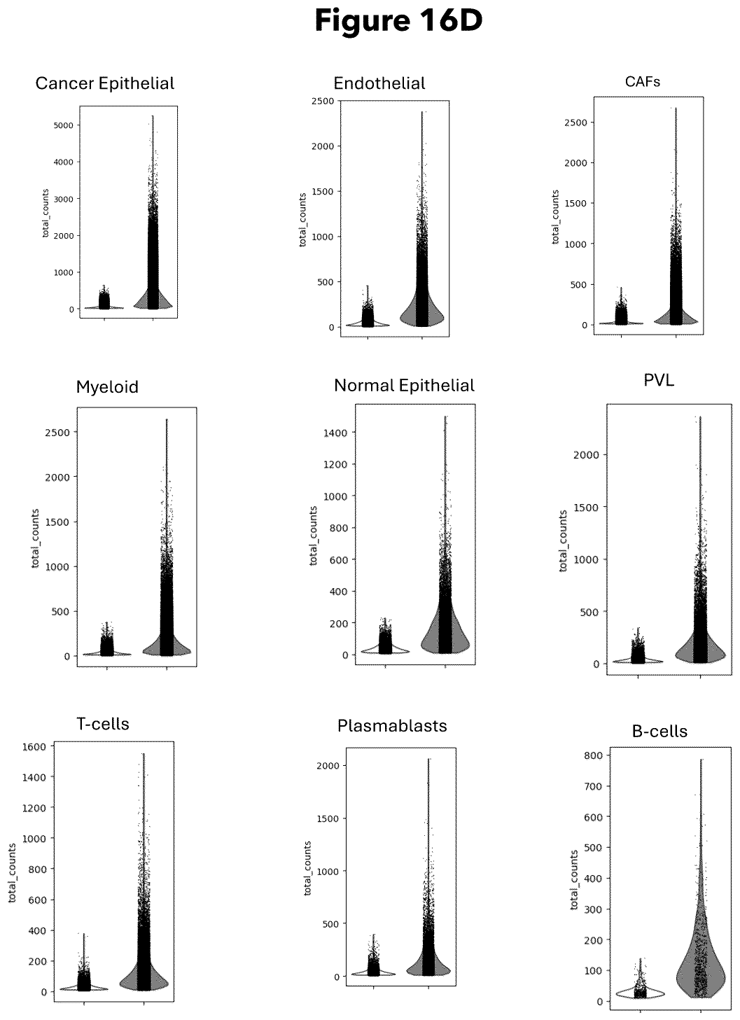

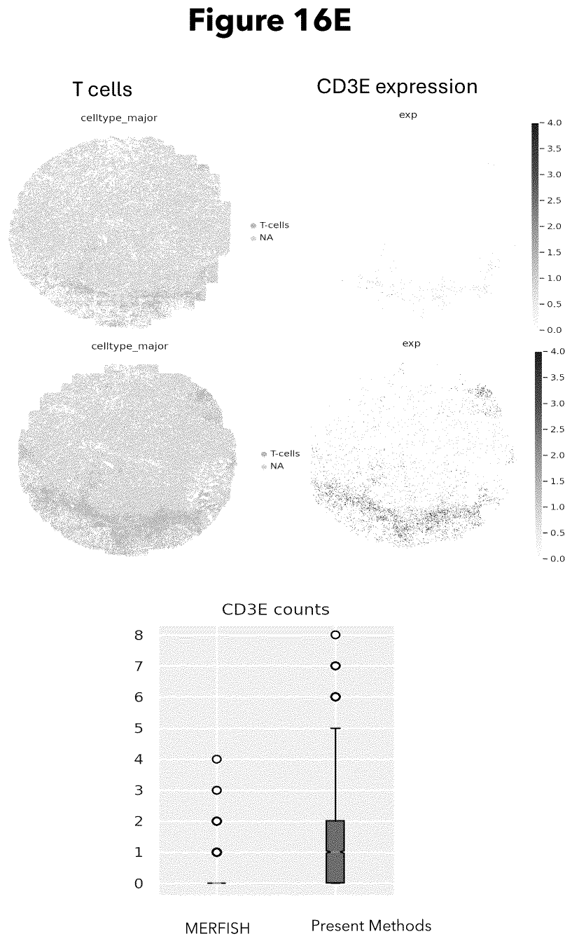

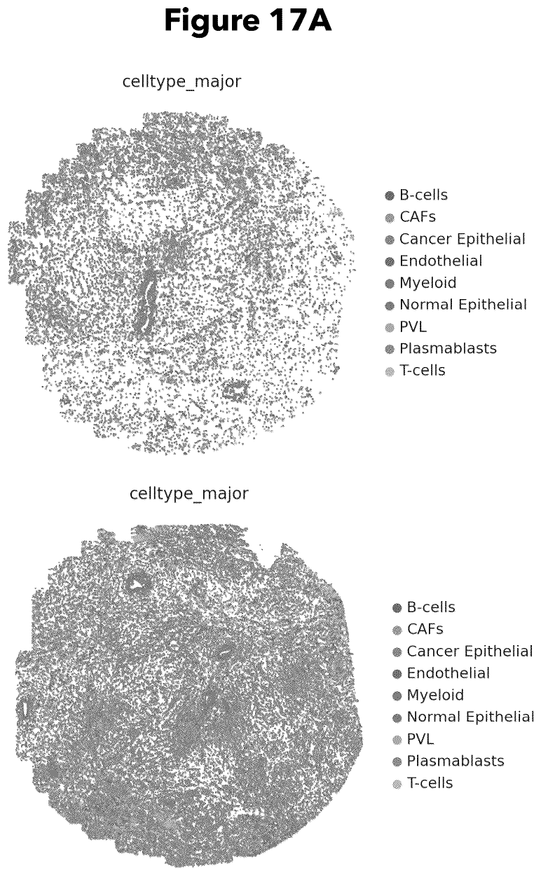

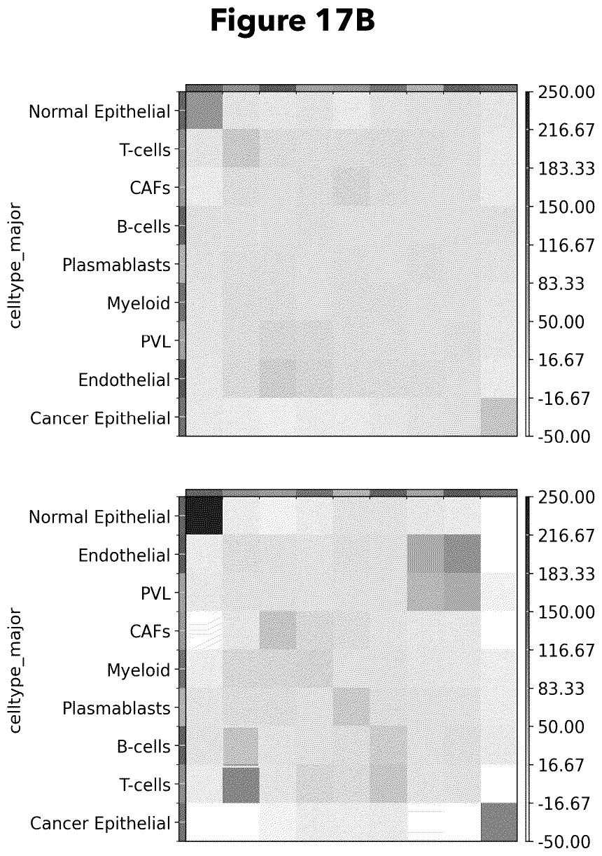

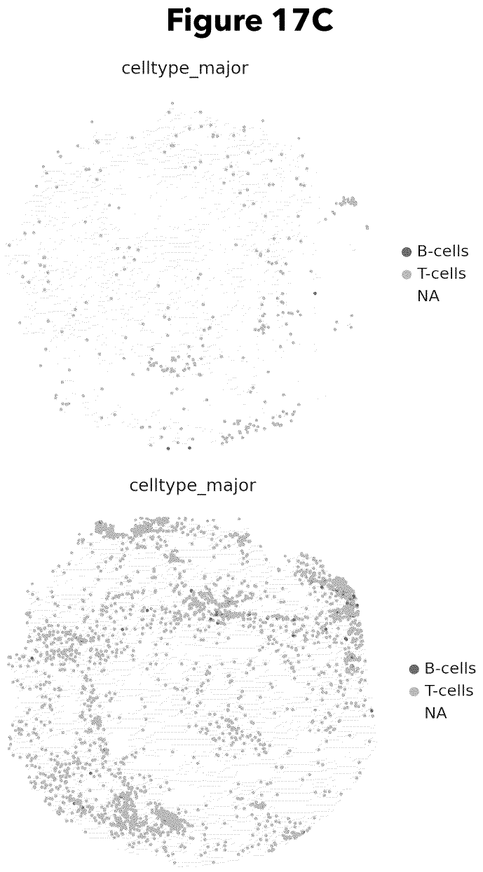

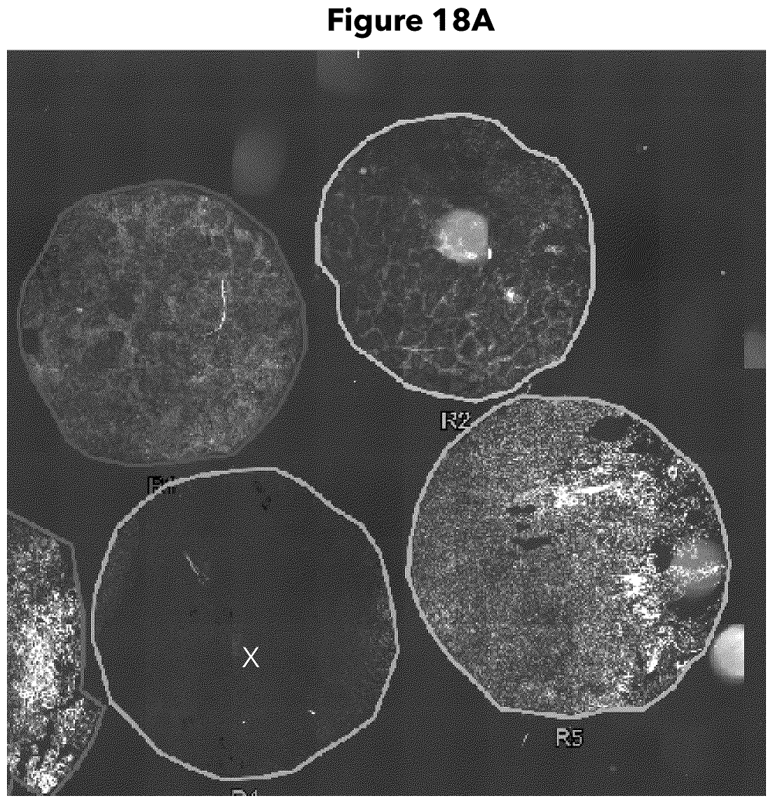

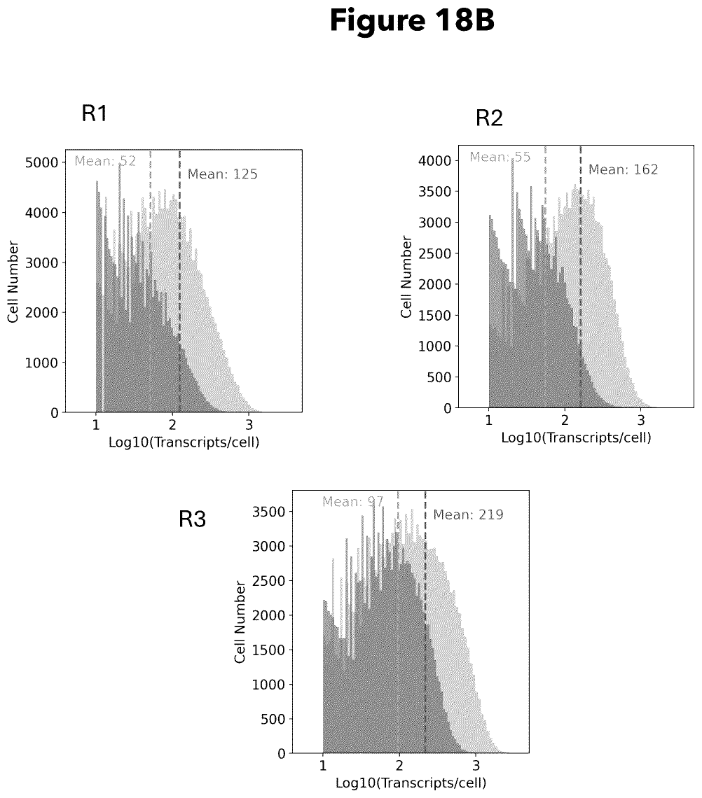

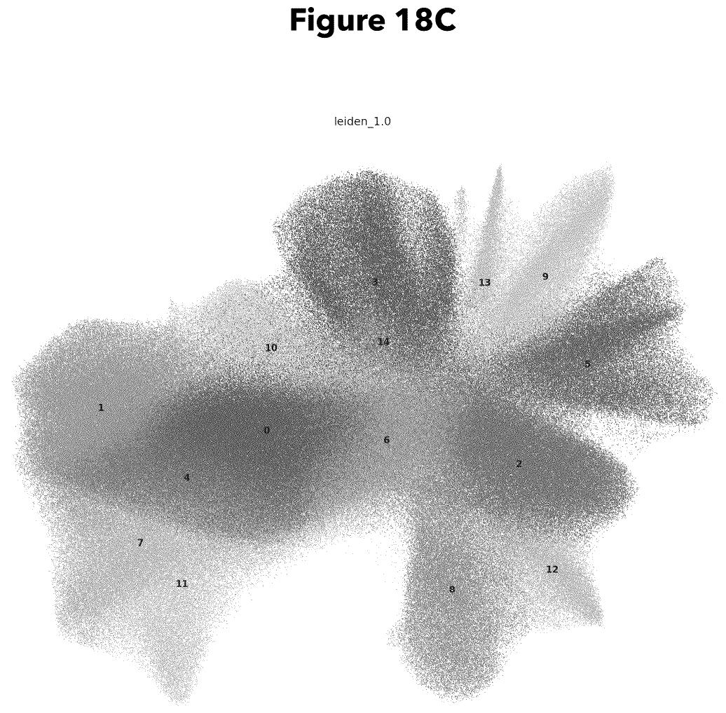

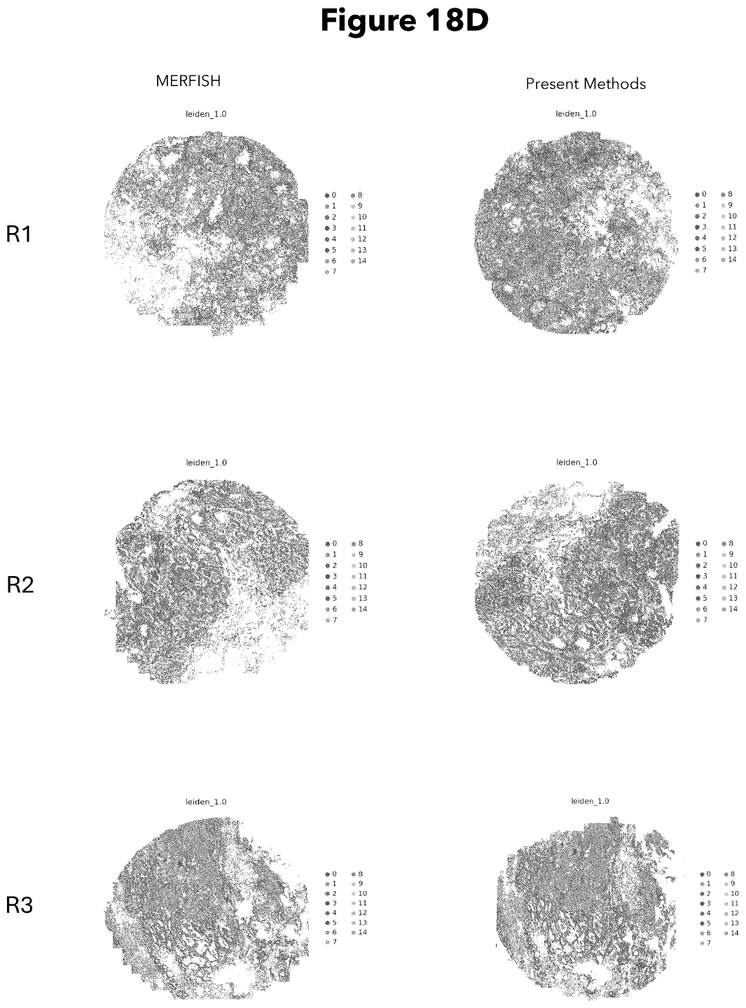

The Applicant herein demonstrates the present primary nucleic acid probes comprising (i) a target binding sequence and (ii) one or more converter sequences complementary to a sandwich probe and the sandwich probes comprising two or more read sequences and optionally an in situ fiducial binding site, wherein the sandwich probes hybridize to the converter sequences provide improved imaging and detection of RNA species in situ. See FIGS. 7-18.

In embodiments, the present disclosure generally relates to compositions and methods for determining a plurality of nucleic acid targets in a sample. In certain aspects, the present disclosure provides primary nucleic acid probes and methods thereof, comprising one or more target sequences configured to hybridize with a distinct nucleic acid species in a sample and one or more converter sequences configured to hybridize to a sandwich probe; a sandwich probe comprising one or more read sequences and optionally an in situ fiducial binding site, one or more read sequences configured to hybridize with at least one readout probe comprising one or more signaling entities. In certain aspects, the present disclosure provides primary nucleic acid probes for methods for multiplexed error robust fluorescence in situ hybridization (MERFISH), and methods for generating fiducial signals for multiplexed error robust fluorescence in situ hybridization (MERFISH) image alignment in a sample. The methods of the present disclosure can be used for the preparation of gene expression profiles of tissue samples. Other aspects of the present disclosure are generally directed to systems or kits involving such methods or the like.

In embodiments provided herein are methods for generating a in situ fiducial signals for image alignment in a sample. This is advantageous with methods of the present disclosure that use sequential rounds of imaging. In embodiments, the methods comprise contacting the sample comprising a plurality of distinct nucleic acid targets in situ with a plurality of primary nucleic acid probes each nucleic acid probe comprising (i) a target binding sequence and (ii) one or more converter sequences complementary to a sandwich probe, and wherein each subpopulation of nucleic acid probes hybridizes to a distinct nucleic acid target; contacting the sample with a plurality of sandwich probes comprising an in situ fiducial binding site wherein the sandwich probes hybridize to the converter sequences; contacting the sample with a plurality of fiducial probes comprising a fluorescent label, wherein the fiducial probes in situ fiducial binding site; and, imaging the readout probes hybridized to the read sequences; providing a reference signal for each nucleic acid target in the sample.

In embodiments, the biological sample may be immobilized or embedded within a polymer or a gel, partially or completely. In some cases, the sample may be embedded within a relatively large polymer or gel, which can then be sectioned or sliced in some cases to produce smaller portions for analysis, e.g., using various microtomy techniques commonly available to those of ordinary skill in the art. For instance, tissues or organs may be immobilized within a suitable polymer or gel. In embodiments, the polymer may be selected to be relatively optically transparent. The polymer may also be one that does not significantly distort during the polymerization process, although in some cases, the polymer may exhibit some distortion. In some cases, the amount of distortion may be determined as a relative change in size that is less than 5, less than 4, less than 3, less than 2, less than 1.5, less than 1.3, or less than 1.2 (i.e., a change in size of 2 means that a sample doubles in linear dimension), or inverses of these (i.e., an inverse change in size of 2 means that a sample halves in linear dimensions).

Examples of suitable polymers include polyacrylamide and agarose. In some cases, the polymer is a gel or a hydrogel. A variety of polymers could be used in various embodiments that involve chemical cross links between gel subunits, including but not limited to acrylic acid, acrylamide, ethylene glycol diacrylate, ethylene glycol dimethacrylate, poly(ethylene glycol dimethacrylate); and/or hydrophobic or hydrogen bonding interactions, such as poly(N-isopropyl acrylamide), methyl cellulose, (ethylene oxide)-(propylene oxide)-(ethylene oxide terpolymers, sodium alginate, poly(vinyl alcohol), alginate, chitosan, gum Arabic, gelatin, and agarose.

### B. Definitions

Unless defined otherwise, all technical and scientific terms have the same meaning as is commonly understood by one of ordinary skill in the art to which the embodiments disclosed belongs. Certain terms are discussed below or elsewhere in the specification to provide additional guidance to the practitioner in describing the compositions and methods of the disclosure and how to make and use them. The scope or meaning of any use of a term will be apparent from the specific context in which it is used.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Embodiments of various compounds and salts thereof are provided. Where a variable is not specifically recited, the variable can be any option described herein, except as otherwise noted or dictated by context.

In some embodiments, the compound is as described in the appended exemplary, non-limiting claims, or a pharmaceutically acceptable salt, solvate or prodrug thereof.

### Biological Samples

As used herein, “biological sample” herein refers to a collection of cells obtained from a biological source (e.g., cell culture or tissue from a subject). The tissue may contain nucleated cells with chromosomal material. The source of the tissue sample may be solid tissue, as from a fresh, frozen, fixed, FFPE, and/or preserved organ or tissue sample, or biopsy, or aspirate, or blood or any blood constituents, or bodily fluids, such as cerebral spinal fluid, amniotic fluid, peritoneal fluid, or interstitial fluid, or cells from any time in gestation or development of the subject. The tissue sample may also be primary or cultured cells or cell lines, or culture tissues. The tissue sample may contain compounds which are not naturally intermixed with the tissue in nature, such as preservatives, anticoagulants, buffers, fixatives, nutrients, antibiotics, or the like. In some embodiments of the invention, the tissue sample is non-hematologic tissue (i.e., not blood or bone marrow tissue). In embodiments, the biological sample is fresh frozen or fixed frozen tissue sample. In embodiments, the tissue sample used in the present methods is a formalin-fixed paraffin embedded tissue sample.

In embodiments, nucleic acid fragmentation can be evaluated and determined using methods well known in the art. For example, to evaluate sample quality for in situ hybridization, it is informative to determine the RNA quality of a tissue block using RNA Integrity Number (RIN) or DV200 values via commercially available instruments such as the BioAnalyzer or TapeStation platforms. Briefly, RNA from the samples are extracted first and measured on either BioAnalyzer or TapeStation. RIN is expressed in values that range from 1-10, where 1 indicates a sample has shorter and more degraded RNA whereas 10 reflects longer and less degraded RNA. Higher RIN scores will have more intact 18S and 28S RNAs. DV200 reflects the percentage of RNA fragments greater than 200 nucleotides in length in tissue samples. Tissues with lower DV200 percentages have shorter and more degraded RNA; conversely, a higher percentage indicates longer, and less degraded RNA molecules present in the tissue.

In some embodiments, the tissue sample is a tissue section, a clinical smear, or a cultured cell or tissue. In some embodiments, the tissue sample comprises a tissue section. As used herein, “section” of a tissue sample herein refers to a single part or piece of a tissue sample, for example, a thin slice of tissue or cells cut from a tissue sample. It is understood that multiple sections of tissue samples may be taken and subjected to analysis according to the present invention. In some embodiments, the selected portion or section of tissue comprises a homogeneous population of cells. In some embodiments, the selected portion or section of tissue comprises a heterogeneous population of cells. In some embodiments, the selected portion comprises a region of tissue, e.g., the lumen as a non-limiting example. The selected portion can be as small as one cell or two cells, or could represent many thousands of cells, for example.

Any tissue sample from the subject may be used. Examples of tissue samples that may be used include, but are not limited to, breast, prostate, ovary, colon, lung, endometrium, stomach, salivary gland, or pancreas. The tissue sample can be obtained by a variety of procedures including, but not limited to, surgical excision, aspiration, or biopsy. The tissue may be fresh frozen, fixed frozen or FFPE.

In some embodiments, the tissue section is a tissue section of brain, adrenal glands, colon, small intestines, stomach, heart, liver, skin, kidney, lung, pancreas, testis, ovary, prostate, uterus, thyroid, and spleen of a mammal (e.g., human or mouse). The methods of the present disclosure may be applied to any type of tissue, including, for example, cancer tissue (including from any cancer). In some embodiments, the tissue section is from a solid tumor. In some embodiments, the tissue sample is from mouse small intestine. In some embodiments, the tissue sample is from mouse brain. In some embodiments, the tissue sample is from human liver cancer. In some embodiments, the tissue sample is from human kidney. In some embodiments, the tissue sample is from human lung. In some embodiments, the tissue sample is from human ovarian cancer. In some embodiments, the tissue sample is from human uterus cancer. In some embodiments, the tissue sample is from human lung cancer.

In some embodiments, the tissue has been stored for a period of time, for example, the period of time that frozen or FFPE are stored. In some embodiments, the tissue sample is a frozen tissue sample. In some embodiments, the tissue is frozen tissue. In some embodiments, the tissue is paraffin-embedded tissue. In some embodiments, the tissue is formalin-fixed paraffin-embedded tissue.

### 1. Preparation of Tissue Sample

### Obtaining and Fixing Tissue Samples

Tissue samples can be obtained from an intact organ or tissue using any methods well known to those of skill in the art, e.g., the prior methods used to prepare tissue samples for immunohistochemistry (IHC) or in situ hybridization (ISH) techniques.

For example, any intact organ or tissue may be cut into reasonably small piece(s) (the size of the cut pieces typically ranges from a few millimeters to a few centimeters) and “fixed” to preserve the positions of the nucleic acids within the sample. Techniques for fixing cells and tissues are known to those of ordinary skill in the art. Non-limiting examples of fixatives include such as formaldehyde, paraformaldehyde, glutaraldehyde, ethanol, methanol, acetone, acetic acid, or the like.

In some embodiments, the tissue sample is fixed in a solution containing an aldehyde. In some embodiments, the tissue sample is fixed in a solution containing formalin. In some embodiments, the tissue sample is paraffin embedded. In embodiments, the tissue sample is both formalin-fixed and paraffin-embedded (FFPE).

In addition to intact samples, other samples may be used. In some embodiments, the frozen-sections may be prepared by rehydrating 50 mg of frozen pulverized tissue at room temperature in phosphate-buffered saline (PBS) in a small plastic capsule; pelleting the particles by centrifugation; resuspending the particles in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections. Similarly, permanent tissue sections may be prepared involving rehydration of the 50 mg sample in a plastic microfuge tube; pelleting; resuspending in 10% formalin for a 4 hour fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

In some embodiments, the present invention may utilize standard frozen samples, such as those that are embedded in OCT and that are not pulverized, for example, including those used in standard Frozen Section hospital labs.

Tissue samples are often fixed by conventional methodology. Aldehyde fixatives such as formalin (formaldehyde) and glutaraldehyde are typically used. Tissue samples fixed using other fixation techniques, such as alcohol immersion, are also suitable. See Battifora and Kopinski, J., Histochem. Cytochem., 34:1095 (1986). One of skill in the art will appreciate that the choice of the fixative is determined by the purpose for which the tissue is to be histologically stained or otherwise analyzed. One of skill in the art will also appreciate that the length of fixation depends upon the size of the tissue sample and the fixative used.

The samples used may also be embedded in paraffin. In some embodiments, the tissue sample is fixed and embedded in paraffin or the like. In some embodiments, the tissue sample is both formalin-fixed and paraffin-embedded. In some embodiments, the formalin-fixed paraffin-embedded (FFPE) tissue block is hematoxylin and eosin (H&E) stained. As commonly known in the art, the tissue sample may be first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology. Examples of paraffin that may be used include, but are not limited to, Paraplast, Broloid, and Tissuemay. Once the tissue sample is embedded, the sample may be sectioned by a microtome or the like. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

In some embodiments, the tissue section may range from about 3 μm to about 100 μm, or any intermediate ranges therewithin. In some embodiments, the tissue section may range from about 10 μm to about 100 μm. In some embodiments, the tissue section may range from about 10 μm to about 50 μm. In some embodiments, the tissue section may range from about 10 μm to about 30 μm. In some embodiments, the tissue section may range from about 10 μm to about 15 μm. In some embodiments, the tissue section may range from about 3 μm to about 15 μm. In some embodiments, the tissue section may range from about 5 μm to about 20 μm. In some embodiments, the tissue section may range from about 15 μm to about 30 μm. In some embodiments, the tissue section may range about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, or about 20 μm. In some embodiments, the tissue section may range about 30 μm, about 40 μm, about 50 μm, about 60 μm, about 70 μm, about 80 μm, about 90 μm, or about 100 μm.

Tissue sections can be deparaffinized using methods known in the art and/or commercially available kits. The methods remove the bulk of paraffin from the sample. Various techniques are known for deparaffinizing and include, but are not limited to, washing with an organic solvent or agent to dissolve the paraffin.

Exemplar deparaffinization solvents include but are not limited to, benzene, toluene, ethylbenzene, xylenes, D-limonene, octane, and mixtures thereof. In certain embodiments, the deparaffinization solvents comprise D-limonene. These solvents are preferably of high purity, usually greater than 99%. The volume used and the number of washes necessary will depend on the size of the sample and the amount of paraffin to be removed. A sample may be washed between 1 and about 10 times, or between about two and about four times. A typical volume of organic solvent is about 500 ml for a 10 mm tissue sample.

After deparaffinization, samples may be rehydrated such as by stepwise washing with aqueous lower alcoholic solutions of decreasing concentrations. Ethanol is a preferred lower alcohol for rehydrations while other alcohols may also be used. Non-limiting examples include methanol, isopropanol, and other C1-C5 alcohols. The sample is alternatively vigorously mixed with alcoholic solutions followed by its removal. In some embodiments, deparaffinization and rehydration are carried out simultaneously using a reagent such as EZ-DEWAX™ (BioGenex, San Ramon, CA), for example.

In some embodiments, the concentration of alcohol is stepwise lowered. In some embodiments, the concentration range of alcohol is decreased stepwise from about 100% to about 70% in water over about three to five incremental steps. In some embodiments, the concentration range of alcohol is decreased stepwise over three incremental steps with 100%, 90%, and 70% respectively.

In some embodiments of the present disclosure, the samples may be pretreated, such as to facilitate directly or indirectly the methods of the invention. In some embodiments, pretreatment of the tissue increases availability of the target nucleic acid or other targets (e.g., for cell morphology staining). Pretreatments for making targets available (e.g., “antigen retrieval” that retrieves or unmasks the biological markers of interest). An extensive review of antigen retrieval may be found in Shi et al. 1997, J Histochem Cytochem, 45 (3): 327. Antigen retrieval includes a variety of methods by which the availability of the target for interaction with a specific detection reagent is maximized.

The most common techniques are protease-induced epitope retrieval (PIER) or heat induced epitope retrieval (HIER). Protease-induced epitope retrieval (PIER) may employ enzymes such as proteinase K, pepsin, trypsin, protease, and any subtypes thereof, in an appropriate buffer to restore the epitope for antibody binding. Heat-induced epitope retrieval (HIER) may employ heat to reverse some cross-links and allow the restoration of epitopes. Citrate buffers, Tris, and EDTA base may be employed as exemplary heat-induced reagents in appropriately pH stabilized manner (e.g., 10 mM sodium citrate, 6.0 pH; 1 mM EDTA, pH 8.0; 10 mM Tris base, 1 mM EDTA solution, 0.05% Tween 20, pH 9.0). Detergents (e.g., Tween 20) may be added to the HIER buffer to increase the epitope retrieval. In certain aspects, many proprietary formulations may be available for the PIER or HIER mediate antigen retrieval.

Selective staining may be conducted on a tissue section for detection of biological markers and identification of cell types (e.g., nuclear and/or cell morphology stains). To facilitate the specific recognition of biological markers in fixed tissue (e.g., FFPE tissue sample post-deparaffinization and rehydration), it is often necessary to retrieve or unmask the biological markers of interest, through “antigen retrieval” (also called epitope retrieval or antigen unmasking)

It is understood that immobilization of the cellular nucleic acid is a multi-step process, wherein a first and/or second anchoring agents are first added to the tissue sample and a covalent bond is formed between the first anchoring agent and target nucleic acid (as disclosed herein for the first anchor agent of the methods) and the second anchoring agent comprises an oligonucleotide that hybridizes with the target nucleic acid (as disclosed herein for the anchor probe or second anchoring agent of the methods) followed by contact with the polymer matrix wherein both the first and second anchoring agents form covalent bonds with the polymer gel. That entire process immobilizes the target nucleic acid in the polymer gel matrix.

In embodiments, the target nucleic acid-anchoring agents react to form a covalent bond with the polymer gel before, during or after formation of the polymer matrix.

### II. Gel Polymer Matrix

In embodiments, the methods disclosed herein comprise immobilizing the nucleic acid targets (e.g., mRNA) within a polymer matrix.

The biological sample may be embedded within a matrix that immobilizes nucleic acid targets. For instance, the matrix may comprise a gel or a polymer, such as polyacrylamide. Thus, for example, acrylamide and a suitable cross-linker (e.g., N,N′-methylenebisacrylamide) can be added to the sample and polymerized to form a gel. The anchor probes or anchoring agents, if present, may include a portion able to polymerize with the gel (e.g., an acrydite moiety) during the polymerization process, and nucleic acids (e.g., mRNAs containing poly-A tails) may then be able to associate with the anchor probe. In such fashion, the mRNAs may be immobilized to the polyacrylamide gel, cither directly or via hybridization to the anchored bridge probes. In embodiments, DNA and/or RNA molecules may be immobilized to the polyacrylamide gel using bridge probes having substantially complementary portions to the DNA or RNA. In other embodiments, cellular DNA and/or RNA molecules may be physically tangled within the polyacrylamide gel, e.g., due to their length, to immobilize them to the polyacrylamide gel.

The sample may be immobilized or embedded within a polymer or a gel, partially or completely. In some cases, the sample may be embedded within a relatively large polymer or gel, which can then be sectioned or sliced in some cases to produce smaller portions for analysis, e.g., using various microtomy techniques commonly available to those of ordinary skill in the art. For instance, tissues or organs may be immobilized within a suitable polymer or gel.

A variety of polymers may be used in some embodiments. In some cases, the polymer may be selected to be relatively optically transparent. The polymer may also be one that does not significantly distort during the polymerization process, although in some cases, the polymer may exhibit some distortion. In some cases, the amount of distortion may be determined as a relative change in size that is less than 5, less than 4, less than 3, less than 2, less than 1.5, less than 1.3, or less than 1.2 (i.e., a change in size of 2 means that a sample doubles in linear dimension), or inverses of these (i.e., an inverse change in size of 2 means that a sample halves in linear dimensions).

Examples of suitable polymers include polyacrylamide and agarose. In embodiments, the polymer is not a hydrogel and/or does not comprise polymers or monomers that swell or expand. A variety of polymers could be used in various embodiments that involve chemical cross links between gel subunits, including but not limited to acrylic acid, acrylamide, ethylene glycol diacrylate, ethylene glycol dimethacrylate, poly(ethylene glycol dimethacrylate); and/or hydrophobic or hydrogen bonding interactions, such as poly(N-isopropyl acrylamide), methyl cellulose, (ethylene oxide)-(propylene oxide)-(ethylene oxide terpolymers, sodium alginate, poly(vinyl alcohol), alginate, chitosan, gum Arabic, gelatin, and agarose.

### III. Tissue Clearing

After immobilization of either the cellular nucleic acids targets to the gel or the bridge probes (if the cellular nucleic acid was not immobilized with anchoring agents), other cellular components (e.g., non-immobilized nucleic acid) within the sample may be removed or degraded.

By “clearing” a tissue sample or a “cleared” tissue sample, it is meant that the tissue sample is made substantially permeable to light, i.e., transparent, and the optical properties of the sample change to allow more light to pass through the sample. In some embodiments, about 70% or more of the light (e.g., white light, ultraviolet light or infrared light) that is used to illuminate the sample will pass through the sample and illuminate only selected cellular components (e.g., nucleic acids) therein, e.g., 75% or more of the light, 80% or more of the light, 85% or more of the light, 90% or more of the light, 95% or more of the light, 98% or more of the light, e.g. 100% of the light will pass through the specimen. Any treatment known for tissue clearing may be used to clear the tissue sample in the methods described herein, which are further discussed below.

Details of tissue clearing have been further discussed in US Patent Publ. No. 2019/0264270 published Aug. 29, 2019, entitled “Matrix imprinting and clearing,” the content of which is incorporated herein by reference in its entirety. Such clearance may include removal (e.g., physical removal) of cellular components from the sample, and/or degradation within the sample, such that they are no longer as prominent within the background. Degradation may include, for example, chemical degradation, enzymatic degradation, or the like.

In some cases, at least 50%, at least 60%, at least 70%, at least 80%, or at least 90% of the undesired components within the sample may be removed or degraded. Such clearance may include physical removal or degradation of the components (e.g., to smaller components, components that are not fluorescent, etc.). Removal or degradation of such components may decrease background fluorescence or autofluorescence within the sample during analysis.

Multiple clearance steps can also be performed in certain embodiments, e.g., to remove or degrade various undesired components.

For example, enzymes, denaturants, chelating agents, chemical agents, and the like, may break down the proteins into smaller components and/or amino acids. These smaller components may be easier to remove physically, and/or may be sufficiently small or inert such that they do not significantly affect the background. Similarly, lipids may be removed or degraded from the sample using surfactants or the like. In some cases, one or more of these are used, e.g., simultaneously or sequentially. Non-limiting examples of suitable enzymes include proteinases such as proteinase K, proteases or peptidases, or digestive enzymes such as trypsin, pepsin, or chymotrypsin. Non-limiting examples of suitable denaturants include guanidine HCl, acetone, acetic acid, urea, or lithium perchlorate. Non-limiting examples of chemical agents able to denature proteins include solvents such as phenol, chloroform, guanidinium isocyanante, urea, formamide, etc. Non-limiting examples of surfactants include Triton X-100 (polyethylene glycol p-(1,1,3,3-tetramethylbutyl)-phenyl ether), SDS (sodium dodecyl sulfate), Igepal CA-630, or poloxamers. Non-limiting examples of chelating agents include ethylenediaminetetraacetic acid (EDTA), citrate, or polyaspartic acid. In some embodiments, compounds such as these may be applied to the sample to remove or degrade proteins, lipids, and/or other components. For instance, a buffer solution (e.g., containing Tris or tris(hydroxymethyl)aminomethane) may be applied to the sample, then removed.

Non-limiting examples of techniques to remove or degrade RNA include RNA enzymes such as Rnase A, Rnase T, or Rnase H, or chemical agents, e.g., via alkaline hydrolysis (for example, by increasing the pH to greater than 10). Non-limiting examples of systems to remove or degrade sugars or extracellular matrix include enzymes such as chitinase, heparinases, or other glycosylases. Non-limiting examples of systems to remove or degrade lipids include enzymes such as lipidases, chemical agents such as alcohols (e.g., methanol or ethanol), or detergents such as Triton X-100 or sodium dodecyl sulfate. Many of these are readily available commercially. In this way, the background of the sample may be reduced, which may facilitate analysis of the nucleic acid probes or other desired targets, e.g., using fluorescence microscopy, or other techniques as discussed herein.

### Cellular Nucleic Acid Targets

The nucleic acid targets may be, for example, DNA, RNA, or other nucleic acids that are present in a cell within a biological sample.

In some embodiments, the nucleic acid target is RNA. The RNA may be coding and/or non-coding RNA. Non-limiting examples of RNA that may be studied within the cell include mRNA, siRNA, rRNA, miRNA, tRNA, lncRNA, snoRNAs, snRNAs, exRNAs, piRNAs, or the like.

The nucleic acids may be endogenous to the cell or added to the cell. For instance, the nucleic acid may be viral, or artificially created. In some cases, the nucleic acid to be determined may be expressed by the cell.

In some cases, a significant portion of the nucleic acid within the cell may be studied. For instance, in some cases, enough of the RNA present within a cell may be determined so as to produce a partial or complete transcriptome of the cell. In some cases, at least 4 unique mRNA gene transcripts are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of mRNAs may be determined within a cell.

In some cases, the transcriptome of a cell may be determined. It should be understood that the transcriptome generally encompasses all RNA transcript molecules produced within a cell, coding and non-coding, not just coding messenger RNA. Thus, for instance, the transcriptome may also include non-coding rRNA, tRNA, siRNA, miRNA, etc. In some embodiments, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined.

The determination of one or more nucleic acids within the sample may be qualitative and/or quantitative. In addition, the determination may also be spatial, e.g., the position of the nucleic acid within the sample may be determined in two or three dimensions. In some embodiments, the positions, number, and/or concentrations of nucleic acids within the cell (or other sample) may be determined.

### C. Nucleic Acid Probes

Provided herein are primary nucleic acid probes, wherein each nucleic acid probe comprising a target binding sequence, one or more converter sequences complementary to a sandwich probe, wherein each unique converter sequence is assigned to two or more positions in a valid codeword (described in more detail below) assigned to a distinct nucleic acid target. Subpopulations of the primary nucleic acid probes hybridize to the distinct nucleic acid target (described in more detail below) and comprise two or more unique converter sequences that translate to the valid codeword for the distinct nucleic acid target

The primary nucleic acid probes may comprise nucleic acids (or entities that can hybridize to a nucleic acid, e.g., specifically) such as DNA, RNA, LNA (locked nucleic acids), PNA (peptide nucleic acids), or combinations thereof. In some cases, additional components can also be present within the nucleic acid probes, e.g., as discussed below. Any suitable method may be used to introduce nucleic acid probes into a sample.

The nucleic acid probes are added to a biological sample. Certain aspects of the present invention are generally directed to nucleic acid probes that are introduced into a sample. The probes may comprise any of a variety of entities that can hybridize to a nucleic acid, typically by Watson-Crick base pairing, such as DNA, RNA, LNA, PNA, etc., depending on the application. The nucleic acid probe typically contains a target sequence that is able to bind to at least a portion of a target nucleic acid. When introduced into a sample, the nucleic acid probe may be able to bind to a specific target nucleic acid (e.g., an RNA, or other nucleic acids as discussed herein). In some cases, the nucleic acid probes may be determined using signaling entities (e.g., as discussed below), and/or by using secondary nucleic acid probes able to bind to the nucleic acid probes (i.e., to primary nucleic acid probes). The determination of such nucleic acid probes is discussed in detail below.

In some cases, more than one distinct (primary) nucleic acid probe may be applied to a sample, e.g., simultaneously. For example, there may be at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes that are applied to a sample, e.g., simultaneously or sequentially.

In certain embodiments, the primary oligonucleotide probes comprise a target sequence designed to hybridize with (optionally) anchored target nucleic acid. The target sequence may be positioned anywhere within the nucleic acid probe (or primary nucleic acid probe). The target sequence may contain a region that is substantially complementary to a portion of a target nucleic acid. In some cases, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary. In some cases, the target sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the target sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the target sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc. Typically, complementarity is determined on the basis of Watson-Crick nucleotide base pairing.

In some embodiments, the one or more target sequences comprise an oligonucleotide that is at least 2, at least 4, at least 6, at least 8 at least 10, at least 12, at least 14, at least 16, at least 18, at least 20, at least 22, at least 24, at least 26, at least 28, at least 30, at least 32, at least 34, at least 36, at least 38, or at least 40 nucleotides in length. In some embodiments, the one or more target sequences comprise an oligonucleotide that is at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some embodiments, the one or more target sequences comprise an oligonucleotide that at least between 10 and 30 nucleotides in length, between 20 and 40 nucleotides in length, between 5 and 50 nucleotides in length, between 25 and 35 nucleotides in length, between 10 and 200 nucleotides in length, or between 10 and 300 nucleotides in length.

The target sequence of a (primary) nucleic acid probe may be determined with reference to a target nucleic acid suspected of being present within a sample. For example, a target nucleic acid to a protein may be determined using the protein's sequence, by determining the nucleic acids that are expressed to form the protein. In some cases, only a portion of the nucleic acids encoding the protein are used, e.g., having the lengths as discussed above. In addition, in some cases, more than one target sequence that can be used to identify a particular target may be used. For instance, multiple probes can be used, sequentially and/or simultaneously, that can bind to or hybridize to different regions of the same target. Hybridization typically refers to an annealing process by which complementary single-stranded nucleic acids associate through Watson-Crick nucleotide base pairing (e.g., hydrogen bonding, guanine-cytosine and adenine-thymine) to form double-stranded nucleic acid.

The primary nucleic acid probes comprise one or more converter sequences complementary to a sandwich probe and wherein each unique converter sequence (also referred to herein as a “bit pair sequence”) is assigned to two or more positions in a valid codeword assigned to a distinct nucleic acid target. The unique converter sequence is used to transfer the valid codeword that was assigned to the target analyte to the readout of the sandwich probe. For example, a 16-bit valid codebook scheme is used to assign unique 16-bit valid codewords to each distinct target analyte. Every position in that 16-bit codebook has a readout sequence assigned to it. A typical individual codeword would contain only four ‘1’ positions meaning of the 16 possible readout sequences only four are used for each individual codeword and the remaining positions are “0”. Those four readout sequences for any given individual codeword are combined into pairs of two read sequences and constructed into encoding sandwich probes, wherein the sequence that hybridizes to the primary nucleic acid probe corresponds and is unique for that pair of readout sequences.

In embodiments provided herein are methods of imaging a nucleic acid target in a sample using the primary nucleic acid probes of this disclosure. In certain embodiments, methods include contacting the sample comprising a plurality of distinct nucleic acid targets in situ with a plurality of nucleic acid probe subpopulations, wherein each subpopulation of nucleic acid probes hybridizes to a distinct nucleic acid target; contacting the primary nucleic acid probes with a plurality of sandwich probes comprising a sequence that is complementary and hybridizes to the one or more converter sequences of the nucleic acid probe and two or more read sequences; contacting the sandwich probes with a plurality of readout probes comprising a fluorescent label, wherein the readout probes hybridize to the read sequences of the sandwich probes; imaging the readout probes bound to the primary nucleic acid probes; and, repeating above steps in one or more sequential hybridization and imaging rounds until all positions in the valid codeword have been imaged.

In certain embodiments provided herein are methods for imaging RNA spatial organization in a sample comprising contacting the sample comprising a plurality of distinct RNA species in situ with a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct RNA species, and wherein each subpopulation of nucleic acid probes hybridizes to a distinct RNA species; contacting the sample with a plurality of sandwich probes comprising two or more read sequences, wherein the sandwich probes hybridize to the converter sequences; contacting the sample with a plurality of readout probes comprising a fluorescent label, wherein the readout probes hybridize to the read sequences; imaging the readout probes hybridized to the read sequences; and, repeating steps c) and d) in one or more sequential hybridization and imaging rounds until all positions in the valid codeword have been imaged, providing an imaged codeword corresponding to each distinct RNA species in a spatial organization.

In certain embodiments, the primary nucleic acid probe further comprises a universal sequence. For any given pool of population of primary nucleic acid probes, they would all comprise the same universal sequence. In embodiments, the universal sequence is complementary to a fiducial sandwich probe comprise a sequence that is complementary and hybridizes to the universal sequence of the nucleic acid probe and one or more sequences complementary to a fiducial signal amplifier. In embodiments, fiducial signal amplifiers comprise a sequence that is complementary and hybridizes to the fiducial sandwich probes and a plurality of fiducial signaling entity sequences. In embodiments, fluorescent labeled nucleic acid probes comprising a sequence that is complementary and hybridizes to the fiducial signaling entity sequences and imaging. The combination of those probes, starting with the universal sequence on the primary nucleic acid probe provides a reference signal for each primary nucleic acid probed in a sample.

### Sandwich Probes

Provided herein are sandwich probes configured to hybridize to the converter sequence of the primary nucleic acid probe and comprising one or more read sequences corresponding to the “1” positions of any given individual valid codeword. In embodiments, the sandwich probe further comprises an in situ fiducial binding site. In embodiments, the sandwich probe comprises a poly T sequence between the in situ fiducial binding site and a read sequence. See FIG. 4.

The “read” sequences are designed to hybridize with secondary nucleic acid probes (“readout probes”) comprising a label (e.g., fluorescent label). In embodiments, the readout probes are conjugated on both ends of the probe sequence to a fluorescent label. See FIG. 4. However, it should be understood that read sequences are not necessary in all cases. In some embodiments, the nucleic acid probe may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences. The read sequences may be positioned anywhere within the sandwich probe. If more than one read sequence is present, the read sequences may be positioned next to each other, and/or interspersed with other sequences. In certain embodiments, sandwich probes comprise at least two read sequence. In certain other embodiments, the sandwich probes comprise two read sequences (“bit pairs”).

The read sequences may be of any length. If more than one read sequence is used, the read sequences may independently have the same or different lengths. For instance, the read sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc. In some embodiments, the read sequence comprises an oligonucleotide that is at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length

The read sequence may be arbitrary or random in some embodiments. In certain cases, the read sequences are chosen so as to reduce or minimize homology with other components of the sample, e.g., such that the read sequences do not themselves bind to or hybridize with other nucleic acids suspected of being within the sample. In some cases, the homology may be less than 10%, less than 8%, less than 7%, less than 6%, less than 5%, less than 4%, less than 3%, less than 2%, or less than 1%. In some cases, there may be a homology of less than 20 base pairs, less than 18 base pairs, less than 15 base pairs, less than 14 base pairs, less than 13 base pairs, less than 12 base pairs, less than 11 base pairs, or less than 10 base pairs. In some cases, the base pairs are sequential.

In certain embodiments, primary oligonucleotide probes are provided as a pool of probes, wherein each pool of nucleic acid probes hybridize to a distinct target nucleic sequence (e.g., distinct RNA transcript). In embodiments each pool of probes encode, via converter sequences, a N-bit binary code that was assigned to each distinct RNA transcript. In certain embodiments, the N-bit binary code has a Hamming weight of at least 2, at least 4, at least 5, at least 6, at least 7 or at least 8, wherein the Hamming weight value is the number of “1” values in the N-bit code and all other positions are “0”. In embodiments the N-bit binary code has a Hamming weight of at least 2, or at least 4, meaning the code contains two or four “1” bit values, respectively, and the other bit positions are “0”. In embodiments, the N-bit binary code has an N value of 3 to 100, with any value thereof possible. In certain embodiments, the binary code is a 4-bit binary code, a 6-bit binary code, a 8-bit binary code, a 16-bit binary code, a 36-bit binary code, a 50-bit binary code, a 54-bit binary code or a 100-bit binary code, or any combination thereof. Each position of the binary code is either a “0” or a “1”, wherein the binding of secondary probes to the read sequence determines if the hybridization read is “0”, wherein no probe binds, or a “1” wherein secondary probe bound to the read sequence of the primary probe. Sequential hybridization and imaging of the secondary readout probes is performed until each position of the N-bit binary code has been read providing a barcode or codeword for the target nucleic acid (e.g. mRNA sequence).

In one set of embodiments, a population of nucleic acid probes may contain a certain number of read sequences, which may be less than the number of targets of the nucleic acid probes in some cases. Those of ordinary skill in the art will be aware that if there is one signaling entity and n read sequences, then in general 2n−1 different nucleic acid targets may be uniquely identified. However, not all possible combinations need be used. For instance, a population of nucleic acid probes may target 12 different nucleic acid sequences, yet contain no more than 8 read sequences. As another example, a population of nucleic acids may target 140 different nucleic acid species, yet contain no more than 16 read sequences. Different nucleic acid sequence targets may be separately identified by using different combinations of read sequences within each probe. For instance, each probe may contain 1, 2, 3, 4, 5, 6, 7, 8, 9, 10, 11, 12, 13, 14, 15, etc. or more read sequences. In some cases, a population of nucleic acid probes may each contain the same number of read sequences, although in other cases, there may be different numbers of read sequences present on the various probes.

As a non-limiting example, a first nucleic acid probe may contain a first target sequence, a first read sequence, and a second read sequence, while a second, different nucleic acid probe may contain a second target sequence, the same first read sequence, but a third read sequence instead of the second read sequence. Such probes may thereby be distinguished by determining the various read sequences present or associated with a given probe or location, as discussed herein.

In addition, the nucleic acid probes (and their corresponding, complementary sites on the encoding probes), in certain embodiments, may be made using only 2 or only 3 of the 4 bases, such as leaving out all the guanosines (Gs) or leaving out all of the cystosines, (Cs) within the probe. Sequences lacking either Gs or Cs may form very little secondary structure in certain embodiments, and can contribute to more uniform, faster hybridization.

In some embodiments, the nucleic acid probe may contain a signaling entity. It should be understood that signaling entities are not required in all cases, however; for instance, the nucleic acid probe may be determined using secondary nucleic acid probes in some embodiments, as is discussed in additional detail below. Examples of signaling entities that can be used are also discussed in more detail below.

Other components may also be present within a nucleic acid probe as well. For example, in one set of embodiments, one or more primer sequences may be present, e.g., to allow for enzymatic amplification of probes. Those of ordinary skill in the art will be aware of primer sequences suitable for applications such as amplification (e.g., using PCR or other suitable techniques). Many such primer sequences are available commercially. Other examples of sequences that may be present within a primary nucleic acid probe include, but are not limited to promoter sequences, operons, identification sequences, nonsense sequences, or the like.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer is (e.g., is designed to be) complementary to and to hybridize to a target nucleic acid. In some embodiments, a primer is a synthetic primer. In some embodiments, a primer is a non-naturally occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

In addition, the components of the nucleic acid probe may be arranged in any suitable order. For instance, in one embodiment, the components may be arranged in a nucleic acid probe as: primer-read sequences-targeting sequence-read sequences-reverse primer. The “read sequences” in this structure may each contain any number (including 0) of read sequences, so long as at least one read sequence is present in the probe. Non-limiting example structures include:
primer-targeting sequence-read sequences-reverse primer,
primer-read sequences-targeting sequence-reverse primer,
targeting sequence-primer-targeting sequence-read sequences-reverse primer,
targeting sequence-primer-read sequences-targeting sequence-reverse primer,
primer-target sequence-read sequences-targeting sequence-reverse primer,
targeting sequence-primer-read sequence-reverse primer,
targeting sequence-read sequence-primer,
read sequence-targeting sequence-primer,
read sequence-primer-targeting sequence-reverse primer, etc.

In addition, the reverse primer is optional in some embodiments, including in all of the above-described examples.

### D. Detection and Imaging Nucleic Acid Target-Probe Complexes

In embodiments, the nucleic acid probes may be directly determined by determining signaling entities (if present), and/or the nucleic acid probes may be determined by using one or more secondary nucleic acid probes (also referred to herein as readout probes), in accordance with certain aspects of the invention. As mentioned, in some cases, the determination may be spatial, e.g., in two or three dimensions. In addition, in some cases, the determination may be quantitative, e.g., the amount or concentration of a primary nucleic acid probe (and of a target nucleic acid) may be determined. Additionally, the secondary probes may comprise any of a variety of entities able to hybridize a nucleic acid, e.g., DNA, RNA, LNA, and/or PNA, etc., depending on the application. Signaling entities are discussed in more detail below.

A secondary nucleic acid probe may comprise a recognition sequence able to bind to or hybridize with a read sequence of a primary nucleic acid probe. In some cases, the binding is specific, or the binding may be such that a recognition sequence preferentially binds to or hybridizes with only one of the read sequences that are present. The secondary nucleic acid probe may also contain one or more signaling entities. If more than one secondary nucleic acid probe is used, the signaling entities may be the same or different. In embodiments, the secondary nucleic acid probe comprises a fluorescent label and may be referred to herein as a fluorescent secondary nucleic acid probe.

The recognition sequences may be of any length, and multiple recognition sequences may be of the same or different lengths. If more than one recognition sequence is used, the recognition sequences may independently have the same or different lengths. For instance, the recognition sequence may be at least 5, at least 10, at least 15, at least 20, at least 25, at least 30, at least 35, at least 40, or at least 50 nucleotides in length. In some cases, the recognition sequence may be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30, between 20 and 40, or between 25 and 35 nucleotides, etc. In one embodiment, the recognition sequence is of the same length as the read sequence. In addition, in some cases, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to a read sequence of the primary nucleic acid probe.

As mentioned, in some cases, the secondary nucleic acid probe may comprise one or more signaling entities. Examples of signaling entities are discussed in more detail below.

As discussed, in certain aspects of the invention, nucleic acid probes are used that contain various “read sequences.” For example, a population or pool of primary nucleic acid probes may contain certain “read sequences” which can bind certain of the secondary nucleic acid probes, and the locations of the primary nucleic acid probes are determined within the sample using secondary nucleic acid probes, e.g., which comprise a signaling entity. As mentioned, in some cases, a population of read sequences may be combined in various combinations to produce different nucleic acid probes, e.g., such that a relatively small number of read sequences may be used to produce a relatively large number of different nucleic acid probes.

Thus, in some cases, a population (also referred to herein as a “pool”) of primary nucleic acid probes (or other nucleic acid probes) may each contain a certain number of read sequences, some of which are shared between different primary nucleic acid probes such that the total population of primary nucleic acid probes may contain a certain number of read sequences. A population of nucleic acid probes may have any suitable number of read sequences. For example, a population of primary nucleic acid probes may have 1, 2, 3, 4, 5, 6, 7, 8, 9, 10, 11, 12, 13, 14, 15, 16, 17, 18, 19, 20 etc. read sequences. More than 20 are also possible in some embodiments. In addition, in some cases, a population of nucleic acid probes may, in total, have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 24 or more, 32 or more, 40 or more, 50 or more, 60 or more, 64 or more, 100 or more, 128 or more, etc. of possible read sequences present, although some or all of the probes may each contain more than one read sequence, as discussed herein. In addition, in some embodiments, the population of nucleic acid probes may have no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, or no more than two read sequences present. Combinations of any of these are also possible, e.g., a population of nucleic acid probes may comprise between 10 and 15 read sequences in total.

As a non-limiting example of an approach to combinatorially producing a relatively large number of nucleic acid probes from a relatively small number of read sequences, in a population of 6 different types of nucleic acid probes, each comprising one or more read sequences, the total number of read sequences within the population may be no greater than 4. It should be understood that although 4 read sequences are used in this example for ease of explanation, in other embodiments, larger numbers of nucleic acid probes may be realized, for example, using 5, 8, 10, 16, 32, etc. or more read sequences, or any other suitable number of read sequences described herein, depending on the application. If each of the primary nucleic acid probes contains two different read sequences, then by using 4 such read sequences (A, B, C, and D), up to 6 probes may be separately identified. It should be noted that in this example, the ordering of read sequences on a nucleic acid probe is not essential, i.e., “AB” and “BA” may be treated as being synonymous (although in other embodiments, the ordering of read sequences may be essential and “AB” and “BA” may not necessarily be synonymous). Similarly, if 5 read sequences are used (A, B, C, D, and E) in the population of primary nucleic acid probes, up to 10 probes may be separately identified. For example, one of ordinary skill in the art would understand that, for k read sequences in a population with n read sequences on each probe, up to x different probes may be produced, assuming that the ordering of read sequences is not essential; because not all of the probes need to have the same number of read sequences and not all combinations of read sequences need to be used in every embodiment, either more or less than this number of different probes may also be used in certain embodiments. In addition, it should also be understood that the number of read sequences on each probe need not be identical in some embodiments. For instance, some probes may contain 2 read sequences while other probes may contain 3 read sequences.

In some aspects, the read sequences and/or the pattern of binding of nucleic acid probes within a sample may be used to define an error-detecting and/or an error-correcting code, for example, to reduce or prevent misidentification or errors of the nucleic acids. Thus, for example, if binding is indicated (e.g., as determined using a signaling entity), then the location may be identified with a “1”; conversely, if no binding is indicated, then the location may be identified with a “0” (or vice versa, in some cases), when using a pool of primary nucleic acid probes comprising read sequences, wherein each pool each pool of probes encode, via read sequences, a N-bit binary code with a Hamming weight of at least 2 that was assigned to each distinct target nucleic acid (e.g. RNA transcript). Multiple rounds of binding determinations, e.g., using different nucleic acid probes, can then be used to create a “codeword,” e.g., for that spatial location based on binding of the readout probes to the read sequences of the primary probes. In some embodiments, the N-bit binary code may be subjected to error detection and/or correction. For instance, the codewords may be organized such that, if no match is found for a given set of read sequences or binding pattern of nucleic acid probes, then the match may be identified as an error, and optionally, error correction may be applied sequences to determine the correct target for the nucleic acid probes. In some cases, the codewords may have fewer “letters” or positions that the total number of nucleic acids encoded by the codewords, e.g., where each codeword encodes a different nucleic acid.

Such error-detecting and/or the error-correction code may take a variety of forms. A variety of such codes have previously been developed in other contexts such as the telecommunications industry, such as Golay codes or Hamming codes. In some embodiments, the read sequences or binding patterns of the nucleic acid probes are assigned such that not every possible combination is assigned.

For example, if 4 read sequences are possible and a primary nucleic acid probe contains 2 read sequences, then up to 6 primary nucleic acid probes could be identified; but the number of primary nucleic acid probes used may be less than 6. Similarly, for k read sequences in a population with n read sequences on each primary nucleic acid probe, x different probes may be produced, but the number of primary nucleic acid probes that are used may be any number more or less than k. In addition, these may be randomly assigned, or assigned in specific ways to increase the ability to detect and/or correct errors.

As another example, if multiple rounds of nucleic acid probes are used, the number of rounds may be arbitrarily chosen. If in each round, each target can give two possible outcomes, such as being detected or not being detected, up to 2n different targets may be possible for n rounds of probes, but the number of nucleic acid targets that are actually used may be any number less than 2n. For example, if in each round, each target can give more than two possible outcomes, such as being detected in different color channels, more than 2n (e.g., 3n, 4n . . . ) different targets may be possible for n rounds of probes. In some cases, the number of nucleic acid targets that are actually used may be any number less than this number. In addition, these may be randomly assigned, or assigned in specific ways to increase the ability to detect and/or correct errors.

For example, in one set of embodiments, the codewords or nucleic acid probes may be assigned within a code space such that the assignments are separated by a Hamming distance, which measures the number of incorrect “reads” in a given pattern that cause the nucleic acid probe to be misinterpreted as a different valid nucleic acid probe. The Hamming weight refers to the distance between the N-bit binary code assigned to a target and thus each pool of primary oligonucleotide probes as encoded via their read sequences. In embodiments a pool of primary probes may have an assigned N-bit binary code with a Hamming weight of at least 4 and a Hamming weight between pools of 4. In that embodiment, errors can both be detected and corrected. In certain cases, the Hamming distance may be at least 2, at least 3, at least 4, at least 5, at least 6, or the like. In addition, in one set of embodiments, the assignments may be formed as a Hamming code, for instance, a Hamming (7, 4) code, a Hamming (15, 11) code, a Hamming (31, 26) code, a Hamming (63, 57) code, a Hamming (127, 120) code, etc. In another set of embodiments, the assignments may form a SECDED code, e.g., a SECDED (8, 4) code, a SECDED (16, 4) code, a SCEDED (16, 11) code, a SCEDED (22, 16) code, a SCEDED (39, 32) code, a SCEDED (72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above.

For example, a code with the same error correcting properties of the SECDED code may be formed by using only binary words that contain a fixed number of ‘1’ bits, such as 4, to encode the targets. In another set of embodiments, the assignments may represent a subset of the possible values taken from codes described above for the purpose of addressing asymmetric readout errors. For example, in some cases, a code in which the number of ‘1’ bits may be fixed for all used binary words may eliminate the biased measurement of words with different numbers of ‘1’s when the rate at which ‘0’ bits are measured as ‘1’s or ‘1’ bits are measured as ‘0’s are different.

Accordingly, in some embodiments, once the codeword is determined (e.g., as discussed herein), the codeword may be compared to the known nucleic acid codewords. If a match is found, then the nucleic acid target can be identified or determined. If no match is found, then an error in the reading of the codeword may be identified. In some cases, error correction can also be applied to determine the correct codeword, and thus resulting in the correct identity of the nucleic acid target. In some cases, the codewords may be selected such that, assuming that there is only one error present, only one possible correct codeword is available, and thus, only one correct identity of the nucleic acid target is possible. In some cases, this may also be generalized to larger codeword spacings or Hamming distances; for instance, the codewords may be selected such that if two, three, or four errors are present (or more in some cases), only one possible correct codeword is available, and thus, only one correct identity of the nucleic acid targets is possible.

The error-correcting code may be a binary error-correcting code, or it may be based on other numbering systems, e.g., ternary or quaternary error-correcting codes. For instance, in one set of embodiments, more than one type of signaling entity may be used and assigned to different numbers within the error-correcting code. Thus, as a non-limiting example, a first signaling entity (or more than one signaling entity, in some cases) may be assigned as “1” and a second signaling entity (or more than one signaling entity, in some cases) may be assigned as “2” (with “0” indicating no signaling entity present), and the codewords distributed to define a ternary error-correcting code. Similarly, a third signaling entity may additionally be assigned as “3” to make a quaternary error-correcting code, etc.

The contents of each of the following references are incorporated herein by reference: U.S. Pat. No. 11,098,303, entitled “Systems and Methods for Determining Nucleic Acids”; U.S. Pat. No. 10,240,146, entitled “Probe Library Construction”; US Patent Publ. No. 2019-0264270, entitled “Matrix Imprinting and Clearing”; and US Patent Publ. No. 2022-0064697, entitled “Amplification methods and systems for MERFISH and other applications,” for further discussions of Multiplexed Error-Robust Fluorescence In Situ Hybridization (MERFISH) and its examples (e.g., MERFISH probes described herein, signal amplification, determining nucleic acid probes, creating codewords, and error detection and correction, etc.).

As discussed above, in certain aspects, signaling entities are determined, e.g., to determine nucleic acid probes and/or to create codewords. In some cases, signaling entities within a sample may be determined, e.g., spatially, using a variety of techniques. In some embodiments, the signaling entities may be fluorescent, and techniques for determining fluorescence within a sample, such as fluorescence microscopy or confocal microscopy, may be used to spatially identify the positions of signaling entities within a cell. In some cases, the positions of entities within the sample may be determined in two or even three dimensions. In addition, in some embodiments, more than one signaling entity may be determined at a time (e.g., signaling entities with different colors or emissions), and/or sequentially.

In addition, in some embodiments, a confidence level for the identified nucleic acid target may be determined. For example, the confidence level may be determined using a ratio of the number of exact matches to the number of matches having one or more one-bit errors. In some cases, only matches having a confidence ratio greater than a certain value may be used. For instance, in certain embodiments, matches may be accepted only if the confidence ratio for the match is greater than about 0.01, greater than about 0.03, greater than about 0.05, greater than about 0.1, greater than about 0.3, greater than about 0.5, greater than about 1, greater than about 3, greater than about 5, greater than about 10, greater than about 30, greater than about 50, greater than about 100, greater than about 300, greater than about 500, greater than about 1000, or any other suitable value. In addition, in some embodiments, matches may be accepted only if the confidence ratio for the identified nucleic acid target is greater than an internal standard or false positive control by about 0.01, about 0.03, about 0.05, about 0.1, about 0.3, about 0.5, about 1, about 3, about 5, about 10, about 30, about 50, about 100, about 300, about 500, about 1000, or any other suitable value.

In some embodiments, the spatial positions of the entities (and thus, nucleic acid probes that the entities may be associated with) may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities or targets optically, e.g., using fluorescence microscopy, using radioactivity, via conjugation with suitable chromophores, or the like. For example, various conventional microscopy techniques that may be used in various embodiments of the invention include, but are not limited to, epi-fluorescence microscopy, total-internal-reflectance microscopy, highly inclined thin-illumination (HILO) microscopy, light-sheet microscopy, scanning confocal microscopy, scanning line confocal microscopy, spinning disk confocal microscopy, or other comparable conventional microscopy techniques.

In some embodiments, in situ hybridization (ISH) techniques for labeling nucleic acids such as DNA or RNA may be used, e.g., where nucleic acid probes may be hybridized to nucleic acids in samples. These may be performed, e.g., at cellular-scale or single-molecule-scale resolutions. In some cases, the ISH probes comprise, consist essentially of, or yet further consist of RNA, DNA, PNA, LNA, other synthetic nucleotides, or the like, and/or a combination of any of these. The presence of a hybridized probe can be measured, for example, with radioactivity using radioactively labeled nucleic acid probes, immunohistochemistry using, for example, biotin labeled nucleic acid probes, enzymatic chromophore or fluorophore generation using, for example, probes that can bind enzymes such as horseradish peroxidase and approaches such as tyramide signal amplification, fluorescence imaging using nucleic acid probes directly labeled with fluorophores, or hybridization of secondary nucleic acid probes to these primary probes, with the secondary probes detected via any of the above methods.

In some cases, the spatial positions may be determined at super resolutions, or at resolutions better than the wavelength of light or the diffraction limit (although in other embodiments, super resolutions are not required). Non-limiting examples include STORM (stochastic optical reconstruction microscopy), STED (stimulated emission depletion microscopy), NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM (Spectral Precision Distance Microscopy), Photo-Activated Localization Microscopy (PALM), Fluorescence Photoactivation Localization Microscopy (FPALM), LIMON (3D Light Microscopical Nanosizing Microscopy), Super-resolution optical fluctuation imaging (SOFI), or the like. Sec, e.g., U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; or WO 2013/090360, published Jun. 20, 2013, entitled “High Resolution Dual-Objective Microscopy,” by Zhuang, et al., each incorporated herein by reference in their entireties.

In one embodiment, the sample may be illuminated by single Gaussian mode laser lines. In some embodiments, the illumination profiled may be flattened by passing these laser lines through a multimode fiber that is vibrated via piezo-electric or other mechanical means. In some embodiments, the illumination profile may be flattened by passing single-mode, Gaussian beams through a variety of refractive beam shapers, such as the piShaper or a series of stacked Powell lenses. In yet another set of embodiments, the Gaussian beams may be passed through a variety of different diffusing elements, such as ground glass or engineered diffusers, which may be spun in some cases at high speeds to remove residual laser speckle. In yet another embodiment, laser illumination may be passed through a series of lenslet arrays to produce overlapping images of the illumination that approximate a flat illumination field.

In some embodiments, the centroids of the spatial positions of the entities may be determined. For example, a centroid of a signaling entity may be determined within an image or series of images using image analysis algorithms known to those of ordinary skill in the art. In some cases, the algorithms may be selected to determine non-overlapping single emitters and/or partially overlapping single emitters in a sample. Non-limiting examples of suitable techniques include a maximum likelihood algorithm, a least squares algorithm, a Bayesian algorithm, a compressed sensing algorithm, or the like. Combinations of these techniques may also be used in some cases.

In addition, the signaling entity may be inactivated in some cases. For example, in some embodiments, a first secondary nucleic acid probe containing a signaling entity may be applied to a sample that can recognize a first read sequence, then the first secondary nucleic acid probe can be inactivated before a second secondary nucleic acid probe is applied to the sample. If multiple signaling entities are used, the same or different techniques may be used to inactivate the signaling entities, and some or all of the multiple signaling entities may be inactivated, e.g., sequentially or simultaneously.

Inactivation may be caused by removal of the signaling entity (e.g., from the sample, or from the nucleic acid probe, etc.), and/or by chemically altering the signaling entity in some fashion, e.g., by photobleaching the signaling entity, bleaching or chemically altering the structure of the signaling entity, e.g., by reduction, etc.). For instance, in one set of embodiments, a fluorescent signaling entity may be inactivated by chemical or optical techniques such as oxidation, photobleaching, chemically bleaching, stringent washing or enzymatic digestion or reaction by exposure to an enzyme, dissociating the signaling entity from other components (e.g., a probe), chemical reaction of the signaling entity (e.g., to a reactant able to alter the structure of the signaling entity) or the like. For instance, bleaching may occur by exposure to oxygen, reducing agents, or the signaling entity could be chemically cleaved from the nucleic acid probe and washed away via fluid flow.

In some embodiments, various nucleic acid probes (including primary and/or secondary nucleic acid probes) may include one or more signaling entities. If more than one nucleic acid probe is used, the signaling entities may each by the same or different. In certain embodiments, a signaling entity is any entity able to emit light. For instance, in one embodiment, the signaling entity is fluorescent. In other embodiments, the signaling entity may be phosphorescent, radioactive, absorptive, etc. In some cases, the signaling entity is any entity that can be determined within a sample at relatively high resolutions, e.g., at resolutions better than the wavelength of visible light or the diffraction limit. The signaling entity may be, for example, a dye, a small molecule, a peptide or protein, or the like. The signaling entity may be a single molecule in some cases. If multiple secondary nucleic acid probes are used, the nucleic acid probes may comprise the same or different signaling entities.

Non-limiting examples of signaling entities include fluorescent entities (fluorophores) or phosphorescent entities, for example, cyanine dyes (e.g., Cy2, Cy3, Cy3B, Cy5, Cy5.5, Cy7, etc.), Alexa Fluor dyes, Atto dyes, photoswtichable dyes, photoactivatable dyes, fluorescent dyes, metal nanoparticles, semiconductor nanoparticles or “quantum dots”, fluorescent proteins such as GFP (Green Fluorescent Protein), or photoactivatable fluorescent proteins, such as PAGFP, PSCFP, PSCFP2, Dendra, Dendra2, EosFP, tdEos, mEos2, mEos3, PamCherry, PAtagRFP, mMaple, mMaple2, and mMaple3. Other suitable signaling entities are known to those of ordinary skill in the art. See, e.g., U.S. Pat. No. 7,838,302 or WO2015160690A1, each incorporated herein by reference in its entirety. In some cases, spectrally distinct fluorescent dyes may be used.

In one set of embodiments, the signaling entity may be attached to an oligonucleotide sequence via a bond that can be cleaved to release the signaling entity. In one set of embodiments, a fluorophore may be conjugated to an oligonucleotide via a cleavable bond, such as a photocleavable bond. Non-limiting examples of photocleavable bonds include, but are not limited to, 1-(2-nitrophenyl)ethyl, 2-nitrobenzyl, biotin phosphoramidite, acrylic phosphoramidite, diethylaminocoumarin, 1-(4,5-dimethoxy-2-nitrophenyl)ethyl, cyclo-dodecyl (dimethoxy-2-nitrophenyl)ethyl, 4-aminomethyl-3-nitrobenzyl, (4-nitro-3-(1-chlorocarbonyloxyethyl)phenyl)methyl-S-acetylthioic acid ester, (4-nitro-3-(1-thlorocarbonyloxyethyl)phenyl)methyl-3-(2-pyridyldithiopropionic acid) ester, 3-(4,4′-dimethoxytrityl)-1-(2-nitrophenyl)-propane-1,3-diol-[2-cyanoethyl-(N,N-diisopropyl)]-phosphoramidite, 1-[2-nitro-5-(6-trifluoroacetylcaproamidomethyl)phenyl]-ethyl-[2-cyano-ethyl-(N,N-diisopropyl)]-phosphoramidite, 1-[2-nitro-5-(6-(4,4′-dimethoxytrityloxy)butyramidomethyl)phenyl]-ethyl-[2-cyanoethyl-(N,N-diisopropyl)]-phosphoramidite, 1-[2-nitro-5-(6-(N-(4,4′-dimethoxytrityl))-biotinamidocaproamido-methyl)phenyl]-ethyl-[2-cyanoethyl-(N,N-diisopropyl)]-phosphoramidite, or similar linkers. In another set of embodiments, the fluorophore may be conjugated to an oligonucleotide via a disulfide bond. The disulfide bond may be cleaved by a variety of reducing agents such as, but not limited to, dithiothreitol, dithioerythritol, beta-mercaptoethanol, sodium borohydride, thioredoxin, glutaredoxin, trypsinogen, hydrazine, diisobutylaluminum hydride, oxalic acid, formic acid, ascorbic acid, phosphorous acid, tin chloride, glutathione, thioglycolate, 2,3-dimercaptopropanol, 2-mercaptoethylamine, 2-aminoethanol, tris(2-carboxyethyl) phosphine, bis(2-mercaptoethyl) sulfone, N,N′-dimethyl-N,N′-bis(mercaptoacetyl) hydrazine, 3-mercaptoproptionate, dimethylformamide, thiopropyl-agarose, tri-n-butylphosphine, cysteine, iron sulfate, sodium sulfite, phosphite, hypophosphite, phosphorothioate, or the like, and/or combinations of any of these. In another embodiment, the fluorophore may be conjugated to an oligonucleotide via one or more phosphorothioate modified nucleotides in which the sulfur modification replaces the bridging and/or non-bridging oxygen. The fluorophore may be cleaved from the oligonucleotide, in certain embodiments, via addition of compounds such as but not limited to iodoethanol, iodine mixed in ethanol, silver nitrate, or mercury chloride. In yet another set of embodiments, the signaling entity may be chemically inactivated through reduction or oxidation. For example, in one embodiment, a chromophore such as Cy5 or Cy7 may be reduced using sodium borohydride to a stable, non-fluorescence state. In still another set of embodiments, a fluorophore may be conjugated to an oligonucleotide via an azo bond, and the azo bond may be cleaved with 2-[(2-N-arylamino)phenylazo]pyridine. In yet another set of embodiments, a fluorophore may be conjugated to an oligonucleotide via a suitable nucleic acid segment that can be cleaved upon suitable exposure to DNAse, e.g., an exodeoxyribonuclease or an endodeoxyribonuclease. Examples include, but are not limited to, deoxyribonuclease I or deoxyribonuclease II. In one set of embodiments, the cleavage may occur via a restriction endonuclease. Non-limiting examples of potentially suitable restriction endonucleases include BamHI, BsrI, NotI, XmaI, PspAI, DpnI, Mbol, MnlI, Eco571, Ksp6321, DraIII, AhaII, SmaI, MluI, HpaI, ApaI, BclI, BstEII, TaqI, EcoRI, SacI, HindII, HacII, DraII, Tsp509I, Sau3AI, PacI, etc. Over 3000 restriction enzymes have been studied in detail, and more than 600 of these are available commercially. In yet another set of embodiments, a fluorophore may be conjugated to biotin, and the oligonucleotide conjugated to avidin or streptavidin. An interaction between biotin and avidin or streptavidin allows the fluorophore to be conjugated to the oligonucleotide, while sufficient exposure to an excess of addition, free biotin could “outcompete” the linkage and thereby cause cleavage to occur. In addition, in another set of embodiments, the probes may be removed using corresponding “toe-hold-probes,” which comprise the same sequence as the probe, as well as an extra number of bases of homology to the encoding probes (e.g., 1-20 extra bases, for example, 5 extra bases). These probes may remove the labeled readout probe through a strand-displacement interaction.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain embodiments, as discussed in detail below, more than one entity may be used, i.e., entities that are chemically different or distinct, for example, structurally. However, in other cases, the entities may be chemically identical or at least substantially chemically identical.

Another aspect of the invention is directed to a computer-implemented method. For instance, a computer and/or an automated system may be provided that is able to automatically and/or repetitively perform any of the methods described herein. As used herein, “automated” devices refer to devices that are able to operate without human direction, i.e., an automated device can perform a function during a period of time after any human has finished taking any action to promote the function, e.g., by entering instructions into a computer to start the process. Typically, automated equipment can perform repetitive functions after this point in time. The processing steps may also be recorded onto a machine-readable medium in some cases.

For example, in some cases, a computer may be used to control imaging of the sample, e.g., using fluorescence microscopy, STORM or other super-resolution techniques such as those described herein. In some cases, the computer may also control operations such as drift correction, physical registration, hybridization and cluster alignment in image analysis, cluster decoding (e.g., fluorescent cluster decoding), error detection or correction (e.g., as discussed herein), noise reduction, identification of foreground features from background features (such as noise or debris in images), or the like. As an example, the computer may be used to control activation and/or excitation of signaling entities within the sample, and/or the acquisition of images of the signaling entities. In one set of embodiments, a sample may be excited using light having various wavelengths and/or intensities, and the sequence of the wavelengths of light used to excite the sample may be correlated, using a computer, to the images acquired of the sample containing the signaling entities. For instance, the computer may apply light having various wavelengths and/or intensities to a sample to yield different average numbers of signaling entities in each region of interest (e.g., one activated entity per location, two activated entities per location, etc.). In some cases, this information may be used to construct an image and/or determine the locations of the signaling entities, in some cases at high resolutions, as noted above.

### SPECIFIC EMBODIMENTS

Specific Embodiment 1: A composition comprising:
a plurality of primary nucleic acid probes, each nucleic acid probe comprising a target binding sequence, one or more converter sequences complementary to a sandwich probe, wherein each unique converter sequence is assigned to two or more positions in a valid codeword assigned to a distinct nucleic acid target;
wherein subpopulations of the primary nucleic acid probes hybridize to the distinct nucleic acid target and comprise two or more unique converter sequences that translate to the valid codeword for the distinct nucleic acid target.

Specific Embodiment 2: The composition of embodiment 1, wherein each subpopulation of primary nucleic acid probes comprises three or more unique converter sequences.

Specific Embodiment 3: The composition of embodiment 1 or 2, wherein each primary nucleic acid probe comprises two converter sequences flanking the target binding sequence.

Specific Embodiment 4: The composition of any preceding embodiment, wherein the sandwich probe further comprises an in situ fiducial binding site.

Specific Embodiment 5: The composition of any preceding embodiment, wherein the sandwich probe further comprises one of more read sequences.

Specific Embodiment 6: The composition of any preceding embodiment, wherein the sandwich probe comprises a poly T sequence between the read sequence and the in situ fiducial binding site.

Specific Embodiment 7: A method for imaging a nucleic acid target in a sample, comprising:
contacting the sample comprising a plurality of distinct nucleic acid targets in situ with a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct nucleic acid target, and wherein each subpopulation of nucleic acid probes hybridizes to a distinct nucleic acid target;
contacting the sample with a plurality of sandwich probes comprising two or more read sequences, wherein the sandwich probes hybridize to the converter sequences;
a. contacting the sample with a plurality of readout probes comprising a fluorescent label, wherein the readout probes hybridize to the read sequences;
b. imaging the readout probes hybridized to the read sequences; and,
c. repeating steps c) and d) in one or more sequential hybridization and imaging rounds until all positions in the valid codeword have been imaged providing imaged distinct nucleic acid targets.

Specific Embodiment 8: The method of embodiment 7, wherein the nucleic acid target is an RNA species or DNA.

Specific Embodiment 9: The method of embodiment 8, wherein the RNA species is an RNA transcript.

Specific Embodiment 10: The method of embodiment 9, wherein the method comprises determining the transcriptome of a cell.

Specific Embodiment 11: The method of embodiments 7-10, wherein the sandwich probe further comprises an in situ fiducial binding site.

Specific Embodiment 12: The method of embodiments 7-11, wherein the target binding sequence comprises an average length of between 10 and 195 nucleotides.

Specific Embodiment 13: A method for imaging RNA spatial organization in a sample comprising:
contacting the sample comprising a plurality of distinct RNA species in situ with a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct RNA species, and wherein each subpopulation of nucleic acid probes hybridizes to a distinct RNA species;
contacting the sample with a plurality of sandwich probes comprising two or more read sequences, wherein the sandwich probes hybridize to the converter sequences;
contacting the sample with a plurality of readout probes comprising a fluorescent label, wherein the readout probes hybridize to the read sequences;
imaging the readout probes hybridized to the read sequences; and,
repeating steps c) and d) in one or more sequential hybridization and imaging rounds until all positions in the valid codeword have been imaged, providing an imaged codeword corresponding to each distinct RNA species in a spatial organization.

Specific Embodiment 14: The method of embodiment 13, wherein each subpopulation of primary nucleic acid probes comprises three or more unique converter sequences.

Specific Embodiment 15: The method of embodiment 13 or 24, wherein each primary nucleic acid probe comprises two converter sequences flanking the target binding sequence.

Specific Embodiment 16: The method of embodiments 13-15, wherein the sandwich probe further comprises an in situ fiducial binding site.

Specific Embodiment 17: The method of embodiments 13-16, wherein the sandwich probe further comprises one of more read sequences.

Specific Embodiment 18: The method of embodiments 13-17, wherein the sandwich probe comprises a poly T sequence between the read sequence and the in situ fiducial binding site.

Specific Embodiment 19: The method of embodiments 13-18, wherein the target binding sequence comprises an average length of between 10 and 195 nucleotides.

Specific Embodiment 20: The method of embodiments 13-19, wherein the assigned codewords have a Hamming distance equal to or greater than 4 between each of the valid codewords.

Specific Embodiment 21: The method of embodiments 13-20, wherein the codeword has a Hamming weight of 4.

Specific Embodiment 22: The method of embodiments 13-21, wherein the imaged codeword is matched to a valid codeword assigned to a distinct RNA species.

Specific Embodiment 23: The method of embodiments 13-22, wherein the Hamming distance is 4 or greater, and the imaged codewords are matched to valid codewords or discarded.

Specific Embodiment 24: The method of embodiments 13-23, wherein the codeword has a Hamming weight of 4, the Hamming distance is 4 or greater, and the imaged codewords are matched to valid codewords or discarded.

Specific Embodiment 25: The method of embodiments 13-24, wherein the RNA species is an RNA transcript.

Specific Embodiment 26: The method of embodiments 13-25, further comprising determining spatial organization of a transcriptome from a single cell.

Specific Embodiment 27: The method of embodiments 13-26, wherein each primary nucleic acid probe subpopulation comprises at least 10 different primary nucleic acid probes.

Specific Embodiment 28: The method of embodiments 13-27, wherein each primary nucleic acid probe subpopulation comprises at least two distinct converter sequences.

Specific Embodiment 29: The method of embodiments 13-28, wherein each primary nucleic acid probe subpopulation comprises two distinct converter sequences.

Specific Embodiment 30: The method of embodiment 13-29, wherein each primary nucleic acid probe subpopulation comprises at least four distinct converter sequences.

Specific Embodiment 31: The method of embodiments 13-30, wherein each unique converter sequence corresponds to two read sequences.

Specific Embodiment 32: The method of embodiments 13-30, wherein each unique converter sequence encodes two positions in the valid codeword.

Specific Embodiment 33: The method of embodiments 13-32, wherein the primary nucleic acid probes comprise a target binding sequence, with an average length of between 10 and 200 nucleotides, that hybridize the distinct RNA species.

Specific Embodiment 34: The method of embodiments 13-33, wherein each primary nucleic acid probe pool comprises at least 10 different target binding sequences.

Specific Embodiment 35: The method of embodiments 13-34, wherein the readout probes are conjugated to fluorescent labels on each end of the probe sequence.

Specific Embodiment 36: The method of embodiments 13-35, wherein after each hybridization and imaging round, the fluorescent label of the readout probe is quenched to inactivate.

Specific Embodiment 37: The method of embodiments 13-36, wherein after each hybridization and imaging round, the fluorescent label is inactivated by chemically or enzymatically cleaving the fluorescent label from the readout probe.

Specific Embodiment 38: The method of embodiments 13-37, wherein the codeword comprises at least a 16-bit code.

Specific Embodiment 39: The method of embodiments 13-38, wherein the plurality of readout probes comprises at least two distinct fluorescent labels.

Specific Embodiment 40: The method of embodiments 13-39, wherein the plurality of readout probes comprises at least three distinct fluorescent labels.

Specific Embodiment 41: The method of embodiments 13-40, wherein the spatial organization of the distinct RNA species is imaged in 2 dimensions.

Specific Embodiment 42: The method of embodiments 13-41, wherein the spatial organization of the distinct RNA species is imaged in 3 dimensions.

Specific Embodiment 43: The method of embodiments 13-42, further comprising determining abundance for the distinct RNA species.

Specific Embodiment 44: A method for generating in situ fiducial signals for image alignment in a sample, comprising:
contacting the sample comprising a plurality of distinct nucleic acid targets in situ with a plurality of primary nucleic acid probes each nucleic acid probe comprising (i) a target binding sequence and (ii) one or more converter sequences complementary to a sandwich probe, and wherein each subpopulation of nucleic acid probes hybridizes to a distinct nucleic acid target;
contacting the sample with a plurality of sandwich probes comprising an in situ fiducial binding site wherein the sandwich probes hybridize to the converter sequences;
contacting the sample with a plurality of fiducial probes comprising a fluorescent label, wherein the fiducial probes in situ fiducial binding site; and,
imaging the readout probes hybridized to the read sequences; providing a reference signal for each nucleic acid target in the sample

Specific Embodiment 45: The method of embodiment 44, wherein each unique converter sequence is assigned to two or more positions in a valid codeword assigned to the distinct nucleic acid target.

Specific Embodiment 46: The method of embodiment 44 or 45, wherein the sandwich probes comprising two or more read sequences.

Specific Embodiment 47: The method of embodiment 46, further comprising contacting the sample with a plurality of readout probes comprising a fluorescent label, wherein the readout probes hybridize to the read sequences.

Specific Embodiment 48: The method of embodiment 47, further comprising imaging the readout probes hybridized to the read sequences.

Specific Embodiment 49: The method of embodiment 47 and 48, wherein the steps are repeated in one or more sequential hybridization and imaging rounds until all positions in the valid codeword have been imaged providing imaged distinct nucleic acid targets.

### EXEMPLIFICATION

The invention now being generally described, it will be more readily understood by reference to the following examples, which are included merely for purposes of illustration of certain embodiments of the present invention, and are not intended to limit the invention.

### Example 1: Generation of Probe Library

2. Amplify the BS1-NonCleave library using the SOP of the library amplification. Note: in the RT step, use an L26-noncleave-F primer instead of a regular L26-F primer.
3. Prepare BS1-NonCleave mouse brain samples (two samples) using the SOP of FF sample prep.
4. After gel embedding and clearing, incubate the 3×3 adapters (5NT_L26ToV0001T8B2_3x and 5NT_V0001T8B2ToV0000T8B1_3x), 20 nM, according to the standard procedure of 3×3 adapters before

5. For DAPI/PolyT incubation on the bench before loading on MERSCOPE, replace regular 488 nm polyT dye with V0000T8B1_A488 dye at 5 nM (15 ul, 1 uM, into 3 ml hyb base buffer and together with 1 ul of DAPI, 5 mg/ml), RT 10 min→30% formamide wash for 10 min, RT→image buffer+rPCO+RNase inhibitors.

### Example 2: Comparison of Nucleic Acid Probes and Methods of this Disclosure to Known Art Methods (e.g. MERFISH)

The primary nucleic acid probes were designed to code each RNA species (“distinct nucleic acid target”) with a unique binary codeword (“valid codeword”) from a set of Hamming distance 4 (HD 4), Hamming weight 4 (HW 4), binary barcodes. See Xia, et al. PNAS, Spatial transcriptome profiling by MERFISH reveals sub-cellular RNA compartmentalization and cell-cycle dependent gene expression, 2019). Briefly, every barcode in this list is separated by a Hamming distance of at least 4 from all other barcodes, hence allowing one- and two-bit errors to be detected and one-bit errors to be corrected. In addition, each barcode has a constant Hamming weight (i.e., the number of “1” bits in each barcode) of 4 to avoid potential bias in the measurement of different barcodes due to a differential rate of “1” to “0” and “0” to “1” errors. To convert each valid barcode onto a target gene isoform, we constructed a set of 30-nt target regions (“target binding sequence”) configured to hybridize to the target transcript with high specificity. See Moffitt et al, PNAS, High-throughput single-cell gene-expression profiling with multiplexed error-robust fluorescence in situ hybridization, 2016.

The MERFISH gene panel was designed with up to 50 encoding probes and assigning the selected binary codeword onto the corresponding transcript for each RNA species, each encoding probe containing a 30-nt target sequence complementary to one of the 30-nt target regions on the RNA and three 18-nt read bit sequences. The design principle of three-letter read bit sequences (“read sequences”) was described previously. See Moffitt et al, PNAS, High-throughput single-cell gene-expression profiling with multiplexed error-robust fluorescence in situ hybridization, 2016. At a minimal, two encoding probes are required to form the Hamming weight 4 (HW4) codeword.

The primary nucleic acid probes of this disclosure were designed using the same target binding sequences as the MERFISH encoding probes providing a pool of up to 50 primary probes (“subpopulation of primary nucleic acid probes”) for each distinct RNA target assigning the valid binary codeword to the corresponding transcript for each RNA species. However, instead of read sequences, the primary nucleic acid probes each comprised one or two converter sequences (e.g., two 30-nucleotide converter sequences (“converter sequences complementary to an encoding sandwich probe”)) flanking the target region at the 5′ and 3′ end. See FIG. 4.

The sandwich probes comprise a target region configured to hybridize to the converter sequences (“complementary and hybridizes to the one or more converter sequences of the nucleic acid probe”), and two 18-nt read sequences, a flanking region consisting of 5 consecutive thymine nucleotides, and a 25-nt in situ fiducial binding site. In situ fiducial bindings sites are for image registration across sequential imaging rounds. Each readout probe was conjugated with two fluorescent dyes. At a minimal, two primary probes are required to form the Hamming weight 4 (HW4) codeword. See FIG. 4.

Probe target binding region sequences and codebooks for gene panels used in this study are provided in Table 1.

TABLE 1

| | MERFISH | Primary nucleic acid probe |
|---|---|---|
| Tissue type | gene panel | gene panel of this disclosure |
| Fresh or fixed frozen mouse | DM1921 | D2M1763 |
| brain | | |
| Fixed frozen mouse spleen, | DM1922 | D2M1971 |
| heart, lung, small intestine, | | |
| kidney, liver | | |
| Human brain | DM1803 | D2M1769 |
| FFPE breast cancer samples | DM1804 | D2M1774 |
| FFPE lung cancer samples | DP1882 | D2M1883 |

Production of MERFISH and primary nucleic acid probes of this disclosure gene panels and sandwich probes.

Oligo pools for MERFISH primary probes or sandwich probes consisting of pools of individual probes were purchased from oligo vendors including Twist, GenScript, or IDT. The probes were then amplified through a procedure described in previous publications. See Moffitt et al, PNAS, High-throughput single-cell gene-expression profiling with multiplexed error-robust fluorescence in situ hybridization, 2016.

### Histological Procedures for Sample Preparation

Fresh and fixed frozen mouse samples used in this study were all purchased from Jackson laboratory, while human samples were all purchased from BioIVT. Frozen tissue blocks were stored at −80C and FFPE tissue blocks were stored at −20C. For sectioning, frozen tissues were cut into 10 μm thick slices onto MERSCOPE slide by a cryostat (Leica CM3050). FFPE tissues were cut into 5 μm thick slices onto MERSCOPE slide by a microtome (Leica RM2155).

For fresh frozen tissue, following tissue sectioning, tissue slices were fixed with 4% paraformaldehyde in PBS for 15 min, washed with PBS twice, and then permeabilized with 70% ethanol overnight. The samples were either processed for MERFISH sample preparation or stored at 4C for up to one month.

For fixed frozen samples, following tissue sectioning, tissue slices were washed with PBS twice, and then permeabilized with 70% ethanol overnight. The samples were either processed for MERFISH sample preparation or stored at 4C for up to one month.

For FFPE tissue, following tissue sectioning, tissue slices were dried for 20 min at room temperature, followed by further drying for 15 min at 55° C. The tissue sections were then deparaffinized by 500 μl Deparaffinization Buffer (Vizgen 20300112) for 5 min at 55° C. twice, washed with 100% ethanol for 2 min three times, and rehydrated by incubating in 90% ethanol and 70% ethanol, each for 2 min. The rehydrated tissue sections were incubated with 5 ml Decrosslinking Buffer (Vizgen 20300115) at 90° C. for 15 min and cooled on bench for 5 min.

### Cell Boundary Stain

All samples used in this study, except mouse and human brains, were stained for cell boundary after histology using Vizgen's Cell Boundary Kit (10400009) following Vizgen's MERSCOPE FFPE Tissue Sample Preparation User Guide. Briefly, tissue sections were blocked with 100 μl Blocking Buffer (PN 20300012) supplemented with RNase inhibitor (NEB, M0314L) at 1:20 dilution for 1 hour, followed by incubating with 100 μl Cell Boundary Primary Staining Solution for 1 hour. Afterwards, the samples were washed with PBS three times, 5 minutes each, and further incubated with Cell Boundary Secondary Staining Solution for another 1 hour. After washing with PBS three times, the samples were post-fixed with 4% paraformaldehyde in PBS for 15 min, washed with PBS twice before proceeding to RNA anchoring.

### MERFISH for FFPE Samples

### RNA Anchoring

The decrosslinked samples were first washed briefly with Conditioning Buffer (Vizgen 20300116) twice, before incubated with 5 ml Conditioning Buffer at 37° C. for 30 min, and Pre-Anchoring Reaction Buffer (100 μl Conditioning Buffer, 5 μl Pre-Anchoring Activator (Vizgen 20300113), 5 μl RNase inhibitor (NEB, M0314L)) for 2 hours at 37° C. The samples were then stained with cell boundary stain as described above, and washed with Sample Prep Wash Buffer (Vizgen 20300001) briefly, and then incubated with Formamide Wash Buffer (Vizgen 20300002) at 37° C. for 30 min. After aspirating the buffer, each sample was incubated with 100 μL Anchoring Buffer (Vizgen 20300117) at 37° C. overnight.

### Gel Embedding and Tissue Clearing

Gel embedding solution was made with 5 mL of Gel Embedding Premix (Vizgen 20300118), 25 μL 10% ammonium persulfate (Sigma, 09913-100G) and 2.5 μL of TEMED (N,N,N′,N′-tetramethylethylenediamine) (Sigma, T7024-25ML). 20 mm Gel Coverslips (Vizgen 30200004) were cleaned with RNAseZap, 70% ethanol, and covered with 100 μL Gel Slick (VWR, 12001-812). Samples were washed with 3 mL of the Gel Embedding Solution for 1 min. Following the removal of the solution, 100 μL of Gel Embedding Solution was added on top of the sample and sandwiched beneath the Gel Coverslip. Excess gel solution was aspirated, and the samples incubated at room temperature for 1.5 h to allow the gel solution to polymerize. After removing the Gel Coverslip, the samples were incubated with Clearing Solution consisting of 100 μL of Protease K (NEB, P8107S) and 5 mL of Clearing Premix (Vizgen 20300003) at 47° C. overnight first and then at 37° C. for one additional night or more until fully cleared. Samples in Clearing Solution were photobleached using the MERSCOPE Photobleacher (Vizgen 1010003) for 3 hours to remove background fluorescence. A fully detailed, step-by-step instruction on the MERFISH sample prep the full protocol is available at https://vizgen.com/resources/merscope-formalin-fixed-paraffin-embedded-tissue-sample-preparation-user-guide/.

### MERFISH Encoding Probe Hybridization

Clearing solution was removed, and each sample was washed with 5 mL Sample Prep Wash Buffer (Vizgen 20300001) for 5 min three times, followed by 30 min incubation with 5 mL Formamide Wash Buffer (Vizgen 20300002) at 37° C. After aspirating the buffer, the sample was incubated with 100 μl MERFISH Gene Panel Mix at 37° C. for 36-48 hours. Following incubation, tissues were washed twice with 5 mL Formamide Wash Buffer at 47° C. for 30 min.

### Sample Imaging

After aspirating the Formamide Wash Buffer, samples were incubated with 3 mL of DAPI and Poly T Reagent (Vizgen 20300021) for 15 minutes at room temperature, washed for 10 minutes with 5 mL of Formamide Wash Buffer and transferred to 5 mL of Sample Prep Wash Buffer. The imaging activation mix was prepared by adding the Imaging Buffer Activator (Vizgen 20300022) and RNase inhibitor to the imaging buffer. The imaging reagents and processed samples were loaded to the MERSCOPE system (Vizgen 10000001). Following a low-resolution DAPI mosaic at 10× magnification the regions of interest were selected for high-resolution imaging at 60×, as described in Xia et al, PNAS, 2019. Full Instrumentation protocol is available at https://vizgen.com/resources/merscope-instrument/. Data was generated, Cellpose algorithm based on cell boundary and DAPI staining was chosen for cell segmentation and downstream analysis.

### Image Processing

The acquired 60× images were registered to correct for imperfect stage movements between imaging rounds using the signal measured from the in situ fiducial signal and processed to identify the position of each transcript detected within the sample using a pixel based decoding strategy as described in Xia et al, PNAS, 2019.

### MERFISH for Frozen Samples

Frozen tissue samples were sectioned and processed by histological procedures, and then stained for cell boundary using Vizgen's Cell Boundary Kit (10400009) as described above. The sections were later hybridized with MERFISH Gene Panel Mix at 37C incubator for 36-48 hours. Following incubation, the tissues were washed with 5 mL Formamide Wash Buffer at 47C for 30 minutes, twice and embedded into a hydrogel using the Gel Embedding Premix (Vizgen 20300004), ammonium persulfate (Sigma, 09913-100G) and TEMED (N,N,N′,N′-tetramethylethylenediamine) (Sigma, T7024-25ML) from the MERSCOPE Sample Prep Kit (10400012). After the gel mix solution solidified, the samples were cleared with Clearing Solution consisting of 50 uL of Protease K (NEB, P8107S) and 5 mL of Clearing Premix (Vizgen 20300003) at 37C overnight. After removing clearing solution, the sample was stained with DAPI and Poly T Reagent (Vizgen 20300021) for 15 minutes at room temperature, washed for 10 minutes with 5 ml of Formamide Wash Buffer, and then imaged on the MERSCOPE system (Vizgen 10000001).

Present Primary Nucleic Acid Probes and Methods of this Disclosure for Frozen and FFPE Samples

Fresh frozen and FFPE samples were sectioned and processed by histological procedures, and then stained for cell boundary using Vizgen's Cell Boundary Kit (Vizgen 10400118) as described above. After post fixing the sample with 4% PFA for 15 minutes, the tissue sections were incubated with Conditioning Buffer (Vizgen 20300116) supplemented with Rnase inhibitor at room temperature for 15 min, and then Pre-Anchoring Reaction Buffer (Vizgen 20300113) overnight at 37° C. in a humidified chamber. Following anchoring pretreatment, tissue slices were then briefly washed with Sample Prep Wash Buffer (Vizgen, 20300001), and Formamide Wash Buffer for 15 min at 37° C. (Vizgen, 20300002), and then Anchoring Buffer (PN 20300117) at 37° C. for 2 hours. Afterwards, the sample was washed with Sample Prep Wash Buffer briefly and then gel embedded and cleared using the MERSCOPE Tissue Sample Prep Kit (Vizgen, 10400194) overnight at 47° C. After tissue clearing, the sample was treated with MERSCOPE Photobleacher (Vizgen 10100003) for 3 hours, washed with Formamide Wash Buffer at 37° C. for 30 min, and then incubated with Gene Panel Mix consisting of primary nucleic acid probes at 47° C. overnight. The sample was then incubated with sandwich probes at 37° C. overnight, and washed to remove background at 37° C. for 20 minutes, twice. The sample was stained with DAPI and Poly T Reagent for 15 minutes at room temperature, washed for 10 minutes with 5 ml of Formamide Wash Buffer, and then imaged on the MERSCOPE. After image acquisition, the data was processed by MERSCOPE and Cellpose algorithm was used to perform cell segmentation based on cell boundary staining using Cell Bound 3 from Vizgen's Cell Boundary Kit.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A composition comprising:
a plurality of primary nucleic acid probes, each nucleic acid probe comprising a target binding sequence, one or more converter sequences complementary to a sandwich probe, wherein each unique converter sequence is assigned to two or more positions in a valid codeword assigned to a distinct nucleic acid target;
wherein subpopulations of the primary nucleic acid probes hybridize to the distinct nucleic acid target and comprise two or more unique converter sequences that translate to the valid codeword for the distinct nucleic acid target.

2. The composition of claim 1, wherein each subpopulation of primary nucleic acid probes comprises three or more unique converter sequences.

3. The composition of claim 1, wherein each primary nucleic acid probe comprises two converter sequences flanking the target binding sequence.

4. The composition of claim 1, wherein the sandwich probe further comprises an in situ fiducial binding site.

5. The composition of claim 1, wherein the sandwich probe further comprises one of more read sequences.

6. The composition of claim 1, wherein the sandwich probe comprises a poly T sequence between the read sequence and the in situ fiducial binding site.

7. A method for imaging a nucleic acid target in a sample, comprising:
a. contacting the sample comprising a plurality of distinct nucleic acid targets in situ with a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct nucleic acid target, and wherein each subpopulation of nucleic acid probes hybridizes to a distinct nucleic acid target;
b. contacting the sample with a plurality of sandwich probes comprising two or more read sequences, wherein the sandwich probes hybridize to the converter sequences;
c. contacting the sample with a plurality of readout probes comprising a fluorescent label, wherein the readout probes hybridize to the read sequences;
d. imaging the readout probes hybridized to the read sequences; and,
e. repeating steps c) and d) in one or more sequential hybridization and imaging rounds until all positions in the valid codeword have been imaged providing imaged distinct nucleic acid targets.

8. The method of claim 7, wherein the nucleic acid target is an RNA species or DNA.

9. The method of claim 8, wherein the RNA species is an RNA transcript.

10. The method of claim 9, wherein the method comprises determining the transcriptome of a cell.

11. (canceled)

12. (canceled)

13. A method for imaging RNA spatial organization in a sample comprising:
a. contacting the sample comprising a plurality of distinct RNA species in situ with a plurality of primary nucleic acid probe subpopulations, each nucleic acid probe comprising (i) a target binding sequence and (ii) one or more converter sequences complementary to a sandwich probe, wherein each unique converter sequence is assigned to two or more positions in a valid codeword assigned to a distinct RNA species, and wherein each subpopulation of nucleic acid probes hybridizes to a distinct RNA species;
b. contacting the sample with a plurality of sandwich probes comprising two or more read sequences, wherein the sandwich probes hybridize to the converter sequences;
c. contacting the sample with a plurality of readout probes comprising a fluorescent label, wherein the readout probes hybridize to the read sequences;
d. imaging the readout probes hybridized to the read sequences; and,
e. repeating steps c) and d) in one or more sequential hybridization and imaging rounds until all positions in the valid codeword have been imaged, providing an imaged codeword corresponding to each distinct RNA species in a spatial organization.

14. The method of claim 13, wherein each subpopulation of primary nucleic acid probes comprises three or more unique converter sequences.

15. The method of claim 13, wherein each primary nucleic acid probe comprises two converter sequences flanking the target binding sequence.

16. The method of claim 13, wherein the sandwich probe further comprises an in situ fiducial binding site.

17. The method of claim 13, wherein the sandwich probe further comprises one of more read sequences.

18. The method of claim 13, wherein the sandwich probe comprises a poly T sequence between the read sequence and the in situ fiducial binding site.

19. (canceled)

20. The method of claim 13, wherein the assigned codewords have a Hamming distance equal to or greater than 4 between each of the valid codewords.

21. (canceled)

22. (canceled)

23. (canceled)

24. (canceled)

25. The method of claim 13, wherein the RNA species is an RNA transcript.

26. (canceled)

27. (canceled)

28. The method of claim 13, wherein each primary nucleic acid probe subpopulation comprises at least two distinct converter sequences.

29. (canceled)

30. (canceled)

31. (canceled)

32. The method of claim 13, wherein each unique converter sequence encodes two positions in the valid codeword.

33-49. (canceled)
